(19) United States
(12) Patent Application Publication
(10) Pub. No.: US 2023/0348914 A1
(43) Pub. Date: Nov. 2, 2023

(54) COMPOSITIONS AND METHODS FOR TREATMENT OF ACUTE LIVER FAILURE

(71) Applicant: University of Massachusetts, Boston, MA (US)

(72) Inventors: Jun Xie, Shrewsbury, MA (US); Yi Wang, Andover, MA (US); Sha Zhu, Bolton, MA (US); Guangping Gao, Westbororugh, MA (US)

(73) Assignee: University of Massachusetts, Boston, MA (US)

(21) Appl. No.: 17/918,689

(22) PCT Filed: Apr. 13, 2021
(86) PCT No.: PCT/US2021/027097

§ 371 Date: Oct. 13, 2022

Related U.S. Application Data
(60) Provisional application No. 63/009,751, filed on Apr. 14, 2020.

(51) Int. Cl. C12N 15/113; C12N 15/86; A61K 48/00; A61P 1/16

(52) U.S. Cl. CPC C12N 15/1137; C12N 15/86; A61K 48/005; A61P 1/16

(57) ABSTRACT

Aspects of the disclosure relate to isolated nucleic acids encoding one or more inhibitory nucleic acids. In some embodiments, the inhibitory nucleic acids are microRNAs (miRNAs) or artificial microRNAs (amiRNAs). The inhibitory nucleic acids may target one or more genes involved in cytochrome p450 toxicity, for example Slc16a2, Acls5, and Cyb5b. In some embodiments, an inhibitory nucleic acid is a miR-375. The disclosure relates, in some aspects, to methods of reducing cytochrome p450-related toxicity in a cell (e.g., a liver cell) by administering the isolated nucleic acids of the disclosure.

### RELATED APPLICATION

This application is a national stage filing under 35 U.S.C. § 371 of international PCT application PCT/US2021/027097, filed Apr. 13, 2021, which claims priority under 35 U.S.C. § 119(e) to U.S. provisional patent application, U.S. Ser. No. 63/009,751, filed Apr. 14, 2020, the entire contents of each of which are incorporated herein by reference.

### REFERENCE TO A SEQUENCE LISTING SUBMITTED AS A TEXT FILE VIA EFS-WEB

This application contains a Sequence Listing which has been submitted in ASCII format via EFS-Web and is hereby incorporated by reference in its entirety. Said ASCII copy, created on Oct. 12, 2022, is named U012070136US01-SEQ-KZM and is 15,581 bytes in size.

### BACKGROUND

Acetaminophen (APAP) overdose is the leading cause of acute liver failure (ALF) in many countries, including the United States. The high incidence of mortality associated with APAP-ALF can be attributed to its rapid onset and lack of an effective treatment. MicroRNAs (miRs) are small (approximately 18-24 nucleotides in length), non-coding RNA molecules encoded in the genomes of plants and animals. It has been observed that highly conserved microRNAs regulate gene expression by binding to the 3′-untranslated regions (3′-UTR) of specific messenger RNAs.

### SUMMARY

Aspects of the disclosure relate to compositions (e.g., isolated nucleic acids, rAAV vectors, rAAVs, etc.) encoding one or more inhibitory nucleic acids that target one or more genes expressed in hepatocytes. The disclosure is based, in part, on isolated nucleic acids and rAAV vectors encoding miR-375 molecules (e.g., dsRNA, siRNA, shRNA, miRNA, amiRNA, ASOs, DNA or RNA aptamers, etc.) and methods of reducing cytochrome p450 (CYP450)-mediated toxicity (e.g., treating acute liver failure (ALF) in a subject) using the same. In some aspects, the disclosure relates to isolated nucleic acids and rAAV vectors encoding one or more inhibitory nucleic acids (e.g., dsRNA, siRNA, shRNA, miRNA, amiRNA, ASOs, DNA or RNA aptamers, etc.) targeting Slc16A, Cyb5b, Acsl5, or a combination thereof, and methods of treating acute liver failure using the same.

In some aspects, the disclosure provides a method for inhibiting cytochrome p450-mediated toxicity in a cell, the method comprising administering to the cell an isolated nucleic acid encoding one or more (e.g., 1, 2, 3, 4, 5, or more) miR-375 molecules.

In some embodiments, cytochrome p450-mediated toxicity is a result of acetaminophen (APAP) overdose. In some embodiments, a subject has acute liver failure (ALF).

In some embodiments, a cell is in a subject. In some embodiments, a subject is a human.

In some embodiments, an isolated nucleic acid comprises a promoter operably linked to the nucleic acid sequence encoding the one or more miR-375 molecules. A promoter may be a constitutive promoter, inducible promoter, tissue-specific promoter, etc. In some embodiments, a promoter is a RNA pol II promoter. In some embodiments, a promoter is a RNA pol III promoter. In some embodiments, a promoter is a liver-specific promoter, for example a TBG promoter.

In some embodiments, a miR-375 molecule comprises the sequence set forth in SEQ ID NO: 1 or is encoded by the sequence set forth in SEQ ID NO: 2. In some embodiments, a miR-375 molecule comprises a sequence that is at least 70%<80%, 90%, 99% or more identical to the nucleic acid sequence set forth in SEQ ID NO: 1.

In some embodiments, an isolated nucleic acid encoding the miR-375 molecule is an artificial miRNA (amiRNA).

In some embodiments, an isolated nucleic acid is flanked by adeno-associated virus (AAV) inverted terminal repeats (ITRs). In some embodiments, AAV ITRs are AAV2 ITRs. In some embodiments, at least one of the AAV ITRs is a truncated ITR (e.g., mTR or ΔITR).

In some embodiments, an isolated nucleic acid encoding the one or more miR-375 molecules is encapsidated by an AAV capsid protein. An AAV capsid protein may be an AAV1, AAV2, AAV3b, AAV4, AAV5, AAV6, AAV7, AAV8, AAV9, AAVrh.10, AAV.PHP.B, etc. capsid protein. In some embodiments, an AAV capsid protein is an AAV8 capsid protein.

In some embodiments, administration comprises systemic injection to a subject. In some embodiments, administration comprises direct injection to the liver of a subject.

In some embodiments, administration results in a decrease of NAPQI (N-acetyl-p-benzoquinone imine) adducts in the cell (e.g., relative to the level of NAPQI adducts in the cell prior to the administration).

In some embodiments, administration results in a decrease of acetaminophen (APAP) uptake in the liver of the subject (e.g., relative to the level of APAP uptake in the liver of the subject prior to the administration).

In some embodiments, administration results in a decrease of Alanine transaminase (ALT) in the cell (e.g., relative to the level of ALT in the cell prior to the administration). In some embodiments, administration results in a decrease of Alanine transaminase (ALT) in the serum of the subject (e.g., relative to the level of ALT in the serum of the subject prior to the administration).

In some aspects, the disclosure provides an artificial microRNA (amiRNA) comprising a miRNA backbone flanking an isolated nucleic acid encoding a miR-375 molecule. In some embodiments, a miR-375 molecule comprises the sequence set forth in SEQ ID NO: 1 or is encoded by the sequence set forth in SEQ ID NO: 2.

In some aspects, the disclosure provides an isolated nucleic acid encoding an amiRNA as described herein. In some embodiments, an isolated nucleic acid further comprises a promoter operably linked to the nucleic acid sequence encoding the amiRNA. In some embodiments, a promoter is a liver-specific promoter. In some embodiments, the promoter is a TBG promoter. In some embodiments, a nucleic acid sequence encoding a miR-375 amiRNA is flanked by adeno-associated virus (AAV) inverted terminal repeats (ITRs). In some embodiments, the AAV ITRs are AAV2 ITRs. In some embodiments, at least one of the AAV ITRs is a truncated ITR (e.g., mTR or ΔITR).

In some aspects, the disclosure provides a recombinant adeno-associated virus (rAAV) comprising an isolated nucleic acid encoding a miR-375 molecule; and an AAV capsid protein. An AAV capsid protein may be an AAV1, AAV2, AAV3b, AAV4, AAV5, AAV6, AAV7, AAV8, AAV9, AAVrh.10, AAV.PHP.B, etc. capsid protein. In some embodiments, a capsid protein is an AAV8 capsid protein.

In some aspects, the disclosure provides an isolated nucleic acid encoding an artificial miRNA (amiRNA) that targets SLC16a2. In some embodiments, the isolated nucleic acid comprises the sequence set forth in SEQ ID NO: 3 or is encoded by the sequence set forth in SEQ ID NO: 4. In some embodiments, an amiRNA targeting Slc16a2 comprises a sequence that is at least 70%<80%, 90%, 99% or more identical to the nucleic acid sequence set forth in SEQ ID NO: 3.

In some aspects, the disclosure provides a recombinant adeno-associated virus (rAAV) vector comprising an isolated nucleic acid encoding an artificial miRNA (amiRNA) that targets SLC16a2 (e.g., comprising the sequence set forth in SEQ ID NO: 3 or encoded by the sequence set forth in SEQ ID NO: 4), flanked by adeno-associated virus (AAV) inverted terminal repeats (ITRs).

In some embodiments, the AAV ITRs are AAV2 ITRs. In some embodiments, at least one of the AAV ITRs is a truncated ITR (e.g., mTR or ΔITR).

In some aspects, the disclosure provides a recombinant adeno-associated virus (rAAV) comprising the rAAV vector comprising an isolated nucleic acid encoding an artificial miRNA (amiRNA) that targets SLC16a2 (e.g., comprising the sequence set forth in SEQ ID NO: 3 or encoded by the sequence set forth in SEQ ID NO: 4); and an AAV capsid protein. An AAV capsid protein may be an AAV1, AAV2, AAV3b, AAV4, AAV5, AAV6, AAV7, AAV8, AAV9, AAVrh.10, AAV.PHP.B, etc. capsid protein. In some embodiments, the capsid protein is an AAV8 capsid protein.

In some aspects, the disclosure provides a recombinant adeno-associated virus (rAAV) vector comprising an isolated nucleic acid encoding an artificial miRNA (amiRNA) that targets Acsl5 (e.g., comprising the sequence set forth in SEQ ID NO: 5 or encoded by the sequence set forth in SEQ ID NO: 6), flanked by adeno-associated virus (AAV) inverted terminal repeats (ITRs). In some embodiments, an amiRNA targeting Acsl5 comprises a sequence that is at least 70%<80%, 90%, 99% or more identical to the nucleic acid sequence set forth in SEQ ID NO: 5.

In some embodiments, the AAV ITRs are AAV2 ITRs. In some embodiments, at least one of the AAV ITRs is a truncated ITR (e.g., mTR or ΔITR).

In some aspects, the disclosure provides a recombinant adeno-associated virus (rAAV) comprising the rAAV vector comprising an isolated nucleic acid encoding an artificial miRNA (amiRNA) that targets Acsl5 (e.g., comprising the sequence set forth in SEQ ID NO: 5 or encoded by the sequence set forth in SEQ ID NO: 6); and an AAV capsid protein. An AAV capsid protein may be an AAV1, AAV2, AAV3b, AAV4, AAV5, AAV6, AAV7, AAV8, AAV9, AAVrh.10, AAV.PHP.B, etc. capsid protein. In some embodiments, the capsid protein is an AAV8 capsid protein.

In some aspects, the disclosure provides a recombinant adeno-associated virus (rAAV) vector comprising an isolated nucleic acid encoding an artificial miRNA (amiRNA) that targets Cyb5b (e.g., comprising the sequence set forth in SEQ ID NO: 7 or encoded by the sequence set forth in SEQ ID NO: 8), flanked by adeno-associated virus (AAV) inverted terminal repeats (ITRs). In some embodiments, an amiRNA targeting Cyb5b comprises a sequence that is at least 70%<80%, 90%, 99% or more identical to the nucleic acid sequence set forth in SEQ ID NO: 7.

In some embodiments, the AAV ITRs are AAV2 ITRs. In some embodiments, at least one of the AAV ITRs is a truncated ITR (e.g., mTR or ΔITR).

In some aspects, the disclosure provides a recombinant adeno-associated virus (rAAV) comprising the rAAV vector comprising an isolated nucleic acid encoding an artificial miRNA (amiRNA) that targets Cyb5b (e.g., comprising the sequence set forth in SEQ ID NO: 7 or encoded by the sequence set forth in SEQ ID NO: 8); and an AAV capsid protein. An AAV capsid protein may be an AAV1, AAV2, AAV3b, AAV4, AAV5, AAV6, AAV7, AAV8, AAV9, AAVrh.10, AAV.PHP.B, etc. capsid protein. In some embodiments, the capsid protein is an AAV8 capsid protein.

In some aspects, the disclosure provides an isolated nucleic acid comprising an expression cassette encoding two or more artificial miRNAs (amiRNAs), wherein each of the two or more amiRNAs is independently selected from: a miR-375 amiRNA, an amiRNA targeting Slc16a2, an amiRNA targeting Acsl5, and an amiRNA targeting Cyb5b.

In some embodiments, the miR-375 amiRNA comprises the sequence set forth in SEQ ID NO: 1 or is encoded by the sequence set forth in SEQ ID NO: 2. In some embodiments, the amiRNA targeting Slc16a2 comprises the sequence set forth in SEQ ID NO: 3 or is encoded by the sequence set forth in SEQ ID NO: 4. In some embodiments, the amiRNA targeting Acsl5 comprises the sequence set forth in SEQ ID NO: 5 or is encoded by the sequence set forth in SEQ ID NO: 6. In some embodiments, the amiRNA targeting Cyb5b comprises the sequence set forth in SEQ ID NO: 7 or is encoded by the sequence set forth in SEQ ID NO: 8.

In some embodiments, the expression cassette comprises a promoter operably linked to the nucleic acid sequence encoding the two or more amiRNAs. In some embodiments, the promoter is a liver-specific promoter. In some embodiments, the promoter is a TBG promoter.

In some embodiments, the expression cassette is flanked by adeno-associated virus (AAV) inverted terminal repeats (ITRs). In some embodiments, the ITRs are AAV2 ITRs. In some embodiments, at least one of the AAV ITRs is a truncated ITR (e.g., mTR or ΔITR).

In some aspects, the disclosure provides a recombinant adeno-associated virus (rAAV) comprising: the rAAV vector comprising an expression cassette encoding two or more artificial miRNAs (amiRNAs), wherein each of the two or more amiRNAs is independently selected from: a miR-375 amiRNA, an amiRNA targeting Slc16a2, an amiRNA targeting Acsl5, and an amiRNA targeting Cyb5b; and an AAV capsid protein. An AAV capsid protein may be an AAV1, AAV2, AAV3b, AAV4, AAV5, AAV6, AAV7, AAV8, AAV9, AAVrh.10, AAV.PHP.B, etc. capsid protein. In some embodiments, the capsid protein is an AAV8 capsid protein.

In some aspects, the disclosure provides a method for inhibiting cytochrome p450-mediated toxicity in a subject, the method comprising administering to the subject an isolated nucleic acid or rAAV as described herein.

In some embodiments, the disclosure provides a method for treating acute liver failure in a subject, the method comprising administering to the subject an isolated nucleic acid or rAAV as described herein.

In some aspects, the disclosure provides a method for decreasing uptake of acetaminophen (APAP) in the liver of a subject, the method comprising administering to the subject an isolated nucleic acid or rAAV as described herein.

In some aspects, the disclosure provides a method for decreasing ALT in the liver of a subject, the method comprising administering to the subject an isolated nucleic acid or rAAV as described herein.

In some aspects, the disclosure provides a method for decreasing NAPQI (N-acetyl-p-benzoquinone imine) adducts in the liver of a subject, the method comprising administering to the subject an isolated nucleic acid or rAAV as described herein.

### BRIEF DESCRIPTION OF DRAWINGS

FIGS. 1A-1C show liver-specific miR-375 expression in mice inhibits acetaminophen (APAP)-induced liver damage. FIG. 1A shows serum alanine aminotransferase (ALT) levels in acetaminophen overdosed mice for different hours. (Control: TBG-PI Guassia; miR-375: scAAV8-TBG-PI-miR-375 (TBG-miR375)). FIG. 1B shows representative gross images of livers from overdosed mice after 24 hours (injected virus dose: 5E11 genome copy per mice).

FIG. 1C shows representative images of hematoxylin and eosin (H&E) stained liver sections of mice 24 hours after acetaminophen administration (#1, #2, #3 indicate 3 individual animals. Magnification, 10×).

FIGS. 2A-2C show data relating to a protection efficacy comparison among AAV-mediated liver-specific delivery of miR-375, miR-125b, or miR-122 in APAP-ALF model. FIG. 2A shows a schematic representation of the experimental design in APAP-induced ALF model. AAV-miRs were injected 2 weeks before the induction of ALF; animals were fasted over-night before APAP administration. FIG. 2B shows representative images of H&E stained liver sections of mice 24 hours after acetaminophen administration (#1, #2, #3 indicate 3 individual animals. Magnification, 10×). FIG. 2C shows serum ALT levels in APAP overdosed mice at different time points.

FIGS. 3A-3D show TBG-miR375 affects hepatic GSH and APAP metabolism. The levels of hepatic glutathione (GSH; FIG. 3A), serum APAP (FIG. 3B), liver APAP (FIG. 3C), and N-acetyl-p-benzoquinone imine (NAPQI)-protein adduct (FIG. 3D) in acetaminophen-overdosed mice at different time points are shown (Control: TBG-Gluc black dot; TBG-miR375 squares).

FIG. 4 shows a volcano plot from RNA-seq data comparing liver transcriptome change between TBG-miR375 and TBG-Gluc expressing mice (Cytochome P450 (CYP) genes are highlighted on the left; Glutathione-S-transferase (GST) genes are highlighted on the right; solute carrier family (SLC) genes are darker shaded).

FIG. 5 shows qRT-PCR analysis of APAP metabolism genes expression in mice treated with AAV-TBG-miR-375 (right bar) or control AAV-Gluc (left bar). *P<0.05, **P<0.01, ***P<0.001 versus control (two-tailed t-test).

FIG. 6 shows data evaluating the contributions of each individual or combination of miR-375 target genes in APAP-ALF inhibition by AAV-mediated artificial microRNA scaffold housing small interfering RNA (siRNA) for gene silencing (amiR). Serum ALT levels in APAP overdosed mice pre-expressing AAV-TBG-miR-375 or indicating AAV-TBG-amiR vectors.

FIG. 7 shows representative images of H&E stained liver sections of mice as treated in FIG. 6 at 24 hours after APAP administration (#1-#5 indicate 5 individual animals. Magnification, 10×).

FIG. 8 shows representative images of H&E stained liver sections of mice as treated in FIG. 6 at 24 hours after APAP administration (#1-#5 indicate 5 individual animals. Magnification, 10×).

### DETAILED DESCRIPTION

Aspects of the disclosure relate to compositions (e.g., isolated nucleic acids, rAAV vectors, rAAVs, etc.) encoding one or more inhibitory nucleic acids that target one or more genes expressed in hepatocytes. The disclosure is based, in part, on isolated nucleic acids and rAAV vectors encoding miR-375 molecules (e.g., dsRNA, siRNA, shRNA, miRNA, amiRNA, ASOs, DNA or RNA aptamers, etc.) and methods of reducing cytochrome p450 (CYP450)-mediated toxicity (e.g., treating acute liver failure (ALF) in a subject) using the same. In some aspects, the disclosure relates to isolated nucleic acids and rAAV vectors encoding one or more inhibitory nucleic acids (e.g., dsRNA, siRNA, shRNA, miRNA, amiRNA, ASOs, DNA or RNA aptamers, etc.) targeting Slc16A, Cyb5b, Acsl5, or a combination thereof, and methods of treating acute liver failure using the same.

Acute liver failure (ALF), also known as fulminant hepatic failure, as used herein, refers to loss of liver function that occurs rapidly. ALF can be caused by various factors such as hepatitis (e.g., viral induced hepatitis), drugs (e.g., acetaminophen (APAP)), toxins (e.g., mushroom poisoning), autoimmune diseases, metabolic diseases (e.g., Wilson's disease), cancer, or heat stroke. In some embodiments, the ALF is caused by drug overdose (e.g., APAP overdose). Cytochrome P-450 (CYPs) are involved in the metabolism of drugs, chemicals and endogenous substrates. In some embodiments, the hepatic CYPs mediate liver toxicity by activating drugs (e.g., APAP) to toxic metabolites. In some embodiments, binding of toxic metabolites of drugs to CYP leads to the formation of anti-CYP antibodies and immune-mediated hepatotoxicity. In these conditions, enhanced CYP activity is associated with lipid peroxidation and the production of reactive oxygen species, which leads to further cellular membrane and mitochondria damage. In some embodiments, CYP is capable of activating carcinogens (see, e.g., Villeneuve et al., Cytochrome P450 and Liver Diseases, Curr Drug Metab. 2004 June; 5(3):273-82). In some embodiments, reducing CYP expression level and/or activity (e.g., CYP expression and/or activity in hepatocytes) can treat ALF (e.g., APAP overdose induced ALF). In some embodiments, ALF (e.g., APAP overdose induced ALF) is also associated with APAP-metabolizing enzymes responsible for cytotoxic byproduct N-acetyl-p-benzoquinone imine (NAPQI). In some embodiments, ALF (e.g., APAP overdose induced ALF) is also associated with NAPQI-scavenging Glutathione-S-transferases (GST). In some embodiments, ALF (e.g., APAP overdose induced ALF) is also associated with Sulfotransferases (SULT). In some embodiments, increasing GST and/or SULT expression level and/or activity can treat ALF (e.g., APAP overdose induced ALF). In some embodiments, genes associated with APAP metabolism are regulated by inhibitory nucleic acids (e.g., miR-375). In some embodiments, delivering miR-375 to cells (e.g., hepatocytes) can inhibit CYP mediated toxicity. In some embodiments, delivering miR-375 to cells (e.g., hepatocytes) can increase GST expression and/or activity. In some embodiments, delivering miR-375 to cells (e.g., hepatocytes) can increase SULT expression and/or activity. In some embodiments, delivering one or more agents (e.g., nucleic acids capable of regulating expression level and/or activity of miR-375 targets) that regulate downstream targets of miR-375 to cells (e.g., hepatocytes) can treat ALF (e.g., APAP overdose induced ALF). In some embodiments, non-limiting examples of miR-375 target genes associated with ALF (e.g., APAP overdose induced ALF) include Cytochrome B5 Type B (CYB5b), Solute Carrier Family 16 Member 2 (SLC16A2), and Long-Chain Acyl-CoA Synthetase 5 (ACSL5). In some embodiments, delivering one or more inhibitory nucleic acid targeting CYB5b, SLC16A2, ACSL5, or a combination thereof can treat ALF (e.g., APAP overdose induced ALF).

### Isolated Nucleic Acids

In some aspects, the disclosure relates to isolated nucleic acids and rAAVs comprising a transgene, wherein the transgene is a hairpin-forming RNA. Non-limiting examples of hairpin-forming RNA include short hairpin RNA (shRNA), microRNA (miRNA) and artificial microRNA (AmiRNA). In some embodiments, nucleic acids are provided herein that contain or encode the target recognition and binding sequences (e.g., a seed sequence or a sequence complementary to a target) of any one of the inhibitory RNAs (e.g., shRNA, miRNA, AmiRNA) disclosed herein.

A “nucleic acid” sequence refers to a DNA or RNA sequence. In some embodiments, proteins and nucleic acids of the disclosure are isolated. As used herein, the term “isolated” means artificially produced. As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulated by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulated by standard techniques known to those of ordinary skill in the art. As used herein with respect to proteins or peptides, the term “isolated” refers to a protein or peptide that has been isolated from its natural environment or artificially produced (e.g., by chemical synthesis, by recombinant DNA technology, etc.).

Generally, hairpin-forming RNAs are arranged into a self-complementary “stem-loop” structure that includes a single nucleic acid encoding a stem portion having a duplex comprising a sense strand (e.g., passenger strand) connected to an antisense strand (e.g., guide strand) by a loop sequence. The passenger strand and the guide strand share complementarity. In some embodiments, the passenger strand and guide strand share 100% complementarity. In some embodiments, the passenger strand and guide strand share at least 50%, at least 60%, at least 70%, at least 80%, at least 90%, at least 95%, or at least 99% complementarity. A passenger strand and a guide strand may lack complementarity due to a base-pair mismatch. In some embodiments, the passenger strand and guide strand of a hairpin-forming RNA have at least 1, at least 2, at least 3, at least 4, at least 5, at least 6, at least 7 at least 8, at least 9, or at least 10 mismatches. Generally, the first 2-8 nucleotides of the stem (relative to the loop) are referred to as “seed” residues and play an important role in target recognition and binding. The first residue of the stem (relative to the loop) is referred to as the “anchor” residue. In some embodiments, hairpin-forming RNA have a mismatch at the anchor residue.

Hairpin-forming RNAs are useful for translational repression and/or gene silencing via the RNAi pathway. Due to having a common secondary structure, hairpin-forming RNAs share the characteristic of being processed by the proteins Drosha and Dicer prior to being loaded into the RNA-induced silencing complex (RISC). Duplex length amongst hairpin-forming RNAs can vary. In some embodiments, a duplex is between about 19 nucleotides and about 200 nucleotides in length. In some embodiments, a duplex is between about between about 14 nucleotides to about 35 nucleotides in length. In some embodiments, a duplex is between about 19 and 150 nucleotides in length. In some embodiments, hairpin-forming RNA has a duplex region that is 19, 20, 21, 22, 23, 24, 25, 26, 27, 28, 29, 30, 31, 32, or 33 nucleotides in length. In some embodiments, a duplex is between about 19 nucleotides and 33 nucleotides in length. In some embodiments, a duplex is between about 40 nucleotides and 100 nucleotides in length. In some embodiments, a duplex is between about 60 and about 80 nucleotides in length.

In some embodiments, an isolated nucleic acid encodes one or more (e.g., 1, 2, 3, 4, 5, 6, 7, 8, 9, 10, or more) inhibitory nucleic acids, for example dsRNA, siRNA, shRNA, miRNA, artificial microRNAs (AmiRNA), etc.).

In some embodiments, the hairpin-forming RNA is a microRNA (miRNA). A microRNA (miRNA) is a small non-coding RNA found in plants and animals and functions in transcriptional and post-translational regulation of gene expression. A “microRNA” or “miRNA” is a small non-coding RNA molecule capable of mediating transcriptional or post-translational gene silencing. Typically, miRNA is transcribed as a hairpin or stem-loop (e.g., having a self-complementarity, single-stranded backbone) duplex structure, referred to as a primary miRNA (pri-miRNA), which is enzymatically processed (e.g., by Drosha, DGCR8, Pasha, etc.) into a pre-miRNA. The length of a pri-miRNA can vary. In some embodiments, a pri-miRNA ranges from about 100 to about 5000 base pairs (e.g., about 100, about 200, about 500, about 1000, about 1200, about 1500, about 1800, or about 2000 base pairs) in length. In some embodiments, a pri-miRNA is greater than 200 base pairs in length (e.g., 2500, 5000, 7000, 9000, or more base pairs in length.

Pre-miRNA, which is also characterized by a hairpin or stem-loop duplex structure, can also vary in length. In some embodiments, pre-miRNA ranges in size from about 40 base pairs in length to about 500 base pairs in length. In some embodiments, pre-miRNA ranges in size from about 50 to 100 base pairs in length. In some embodiments, pre-miRNA ranges in size from about 50 to about 90 base pairs in length (e.g., about 50, about 52, about 54, about 56, about 58, about 60, about 62, about 64, about 66, about 68, about 70, about 72, about 74, about 76, about 78, about 80, about 82, about 84, about 86, about 88, or about 90 base pairs in length).

Generally, pre-miRNA is exported into the cytoplasm, and enzymatically processed by Dicer to first produce an imperfect miRNA/miRNA* duplex and then a single-stranded mature miRNA molecule, which is subsequently loaded into the RNA-induced silencing complex (RISC). Typically, a mature miRNA molecule ranges in size from about 19 to about 30 base pairs in length. In some embodiments, a mature miRNA molecule is about 19, about 20, about 21, about 22, about 23, about 24, about 25, about 26, about 27, about 28, about 29, or 30 base pairs in length.

In some embodiments, the hairpin-forming RNA is an artificial microRNA (AmiRNA). As used herein “artificial miRNA” or “amiRNA” refers to an endogenous pri-miRNA or pre-miRNA (e.g., a miRNA backbone, which is a precursor miRNA capable of producing a functional mature miRNA), in which the miRNA and miRNA* (e.g., passenger strand of the miRNA duplex) sequences have been replaced with corresponding amiRNA/amiRNA* sequences that direct highly efficient RNA silencing of the targeted gene, for example as described by Eamens et al. (2014), Methods Mol. Biol. 1062:211-224. In some embodiments, the AmiRNA backbone is derived from a pri-miRNA selected from the group consisting of pri-MIR-21, pri-MIR-22, pri-MIR-26a, pri-MIR-30a, pri-MIR-33, pri-MIR-64, pri-MIR-122, pri-MIR-155, pri-MIR-375, pri-MIR-199, pri-MIR-99, pri-MIR-194, pri-MIR-155, and pri-MIR-451.

The following non-limiting list of miRNA genes, and their homologues, which are also useful in certain embodiments of the vectors provided herein: hsa-let-7a, hsa-let-7a*, hsa-let-7b, hsa-let-7b*, hsa-let-7c, hsa-let-7c*, hsa-let-7d, hsa-let-7d*, hsa-let-7e, hsa-let-7e*, hsa-let-7f, hsa-let-7f-1*, hsa-let-7f-2*, hsa-let-7g, hsa-let-7g*, hsa-let-7i, hsa-let-7i*, hsa-miR-1, hsa-miR-100, hsa-miR-100*, hsa-miR-101, hsa-miR-101*, hsa-miR-103, hsa-miR-105, hsa-miR-105*, hsa-miR-106a, hsa-miR-106a*, hsa-miR-106b, hsa-miR-106b*, hsa-miR-107, hsa-miR-10a, hsa-miR-10a*, hsa-miR-10b, hsa-miR-10b*, hsa-miR-1178, hsa-miR-1179, hsa-miR-1180, hsa-miR-1181, hsa-miR-1182, hsa-miR-1183, hsa-miR-1184, hsa-miR-1185, hsa-miR-1197, hsa-miR-1200, hsa-miR-1201, hsa-miR-1202, hsa-miR-1203, hsa-miR-1204, hsa-miR-1205, hsa-miR-1206, hsa-miR-1207-3p, hsa-miR-1207-5p, hsa-miR-1208, hsa-miR-122, hsa-miR-122*, hsa-miR-1224-3p, hsa-miR-1224-5p, hsa-miR-1225-3p, hsa-miR-1225-5p, hsa-miR-1226, hsa-miR-1226*, hsa-miR-1227, hsa-miR-1228, hsa-miR-1228*, hsa-miR-1229, hsa-miR-1231, hsa-miR-1233, hsa-miR-1234, hsa-miR-1236, hsa-miR-1237, hsa-miR-1238, hsa-miR-124, hsa-miR-124*, hsa-miR-1243, hsa-miR-1244, hsa-miR-1245, hsa-miR-1246, hsa-miR-1247, hsa-miR-1248, hsa-miR-1249, hsa-miR-1250, hsa-miR-1251, hsa-miR-1252, hsa-miR-1253, hsa-miR-1254, hsa-miR-1255a, hsa-miR-1255b, hsa-miR-1256, hsa-miR-1257, hsa-miR-1258, hsa-miR-1259, hsa-miR-125a-3p, hsa-miR-125a-5p, hsa-miR-125b, hsa-miR-125b-1*, hsa-miR-125b-2*, hsa-miR-126, hsa-miR-126*, hsa-miR-1260, hsa-miR-1261, hsa-miR-1262, hsa-miR-1263, hsa-miR-1264, hsa-miR-1265, hsa-miR-1266, hsa-miR-1267, hsa-miR-1268, hsa-miR-1269, hsa-miR-1270, hsa-miR-1271, hsa-miR-1272, hsa-miR-1273, hsa-miR-127-3p, hsa-miR-1274a, hsa-miR-1274b, hsa-miR-1275, hsa-miR-127-5p, hsa-miR-1276, hsa-miR-1277, hsa-miR-1278, hsa-miR-1279, hsa-miR-128, hsa-miR-1280, hsa-miR-1281, hsa-miR-1282, hsa-miR-1283, hsa-miR-1284, hsa-miR-1285, hsa-miR-1286, hsa-miR-1287, hsa-miR-1288, hsa-miR-1289, hsa-miR-129*, hsa-miR-1290, hsa-miR-1291, hsa-miR-1292, hsa-miR-1293, hsa-miR-129-3p, hsa-miR-1294, hsa-miR-1295, hsa-miR-129-5p, hsa-miR-1296, hsa-miR-1297, hsa-miR-1298, hsa-miR-1299, hsa-miR-1300, hsa-miR-1301, hsa-miR-1302, hsa-miR-1303, hsa-miR-1304, hsa-miR-1305, hsa-miR-1306, hsa-miR-1307, hsa-miR-1308, hsa-miR-130a, hsa-miR-130a*, hsa-miR-130b, hsa-miR-130b*, hsa-miR-132, hsa-miR-132*, hsa-miR-1321, hsa-miR-1322, hsa-miR-1323, hsa-miR-1324, hsa-miR-133a, hsa-miR-133b, hsa-miR-134, hsa-miR-135a, hsa-miR-135a*, hsa-miR-135b, hsa-miR-135b*, hsa-miR-136, hsa-miR-136*, hsa-miR-137, hsa-miR-138, hsa-miR-138-1*, hsa-miR-138-2*, hsa-miR-139-3p, hsa-miR-139-5p, hsa-miR-140-3p, hsa-miR-140-5p, hsa-miR-141, hsa-miR-141*, hsa-miR-142-3p, hsa-miR-142-5p, hsa-miR-143, hsa-miR-143*, hsa-miR-144, hsa-miR-144*, hsa-miR-145, hsa-miR-145*, hsa-miR-146a, hsa-miR-146a*, hsa-miR-146b-3p, hsa-miR-146b-5p, hsa-miR-147, hsa-miR-147b, hsa-miR-148a, hsa-miR-148a*, hsa-miR-148b, hsa-miR-148b*, hsa-miR-149, hsa-miR-149*, hsa-miR-150, hsa-miR-150*, hsa-miR-151-3p, hsa-miR-151-5p, hsa-miR-152, hsa-miR-153, hsa-miR-154, hsa-miR-154*, hsa-miR-155, hsa-miR-155*, hsa-miR-15a, hsa-miR-15a*, hsa-miR-15b, hsa-miR-15b*, hsa-miR-16, hsa-miR-16-1*, hsa-miR-16-2*, hsa-miR-17, hsa-miR-17*, hsa-miR-181a, hsa-miR-181a*, hsa-miR-181a-2*, hsa-miR-181b, hsa-miR-181c, hsa-miR-181c*, hsa-miR-181d, hsa-miR-182, hsa-miR-182*, hsa-miR-1825, hsa-miR-1826, hsa-miR-1827, hsa-miR-183, hsa-miR-183*, hsa-miR-184, hsa-miR-185, hsa-miR-185*, hsa-miR-186, hsa-miR-186*, hsa-miR-187, hsa-miR-187*, hsa-miR-188-3p, hsa-miR-188-5p, hsa-miR-18a, hsa-miR-18a*, hsa-miR-18b, hsa-miR-18b*, hsa-miR-190, hsa-miR-190b, hsa-miR-191, hsa-miR-191*, hsa-miR-192, hsa-miR-192*, hsa-miR-193a-3p, hsa-miR-193a-5p, hsa-miR-193b, hsa-miR-193b*, hsa-miR-194, hsa-miR-194*, hsa-miR-195, hsa-miR-195*, hsa-miR-196a, hsa-miR-196a*, hsa-miR-196b, hsa-miR-197, hsa-miR-198, hsa-miR-199a-3p, hsa-miR-199a-5p, hsa-miR-199b-5p, hsa-miR-19a, hsa-miR-19a*, hsa-miR-19b, hsa-miR-19b-1*, hsa-miR-19b-2*, hsa-miR-200a, hsa-miR-200a*, hsa-miR-200b, hsa-miR-200b*, hsa-miR-200c, hsa-miR-200c*, hsa-miR-202, hsa-miR-202*, hsa-miR-203, hsa-miR-204, hsa-miR-205, hsa-miR-206, hsa-miR-208a, hsa-miR-208b, hsa-miR-20a, hsa-miR-20a*, hsa-miR-20b, hsa-miR-20b*, hsa-miR-21, hsa-miR-21*, hsa-miR-210, hsa-miR-211, hsa-miR-212, hsa-miR-214, hsa-miR-214*, hsa-miR-215, hsa-miR-216a, hsa-miR-216b, hsa-miR-217, hsa-miR-218, hsa-miR-218-1*, hsa-miR-218-2*, hsa-miR-219-1-3p, hsa-miR-219-2-3p, hsa-miR-219-5p, hsa-miR-22, hsa-miR-22*, hsa-miR-220a, hsa-miR-220b, hsa-miR-220c, hsa-miR-221, hsa-miR-221*, hsa-miR-222, hsa-miR-222*, hsa-miR-223, hsa-miR-223*, hsa-miR-224, hsa-miR-23a, hsa-miR-23a*, hsa-miR-23b, hsa-miR-23b*, hsa-miR-24, hsa-miR-24-1*, hsa-miR-24-2*, hsa-miR-25, hsa-miR-25*, hsa-miR-26a, hsa-miR-26a-1*, hsa-miR-26a-2*, hsa-miR-26b, hsa-miR-26b*, hsa-miR-27a, hsa-miR-27a*, hsa-miR-27b, hsa-miR-27b*, hsa-miR-28-3p, hsa-miR-28-5p, hsa-miR-296-3p, hsa-miR-296-5p, hsa-miR-297, hsa-miR-298, hsa-miR-299-3p, hsa-miR-299-5p, hsa-miR-29a, hsa-miR-29a*, hsa-miR-29b, hsa-miR-29b-1*, hsa-miR-29b-2*, hsa-miR-29c, hsa-miR-29c*, hsa-miR-300, hsa-miR-301a, hsa-miR-301b, hsa-miR-302a, hsa-miR-302a*, hsa-miR-302b, hsa-miR-302b*, hsa-miR-302c, hsa-miR-302c*, hsa-miR-302d, hsa-miR-302d*, hsa-miR-302e, hsa-miR-302f, hsa-miR-30a, hsa-miR-30a*, hsa-miR-30b, hsa-miR-30b*, hsa-miR-30c, hsa-miR-30c-1*, hsa-miR-30c-2*, hsa-miR-30d, hsa-miR-30d*, hsa-miR-30e, hsa-miR-30e*, hsa-miR-31, hsa-miR-31*, hsa-miR-32, hsa-miR-32*, hsa-miR-320a, hsa-miR-320b, hsa-miR-320c, hsa-miR-320d, hsa-miR-323-3p, hsa-miR-323-5p, hsa-miR-324-3p, hsa-miR-324-5p, hsa-miR-325, hsa-miR-326, hsa-miR-328, hsa-miR-329, hsa-miR-330-3p, hsa-miR-330-5p, hsa-miR-331-3p, hsa-miR-331-5p, hsa-miR-335, hsa-miR-335*, hsa-miR-337-3p, hsa-miR-337-5p, hsa-miR-338-3p, hsa-miR-338-5p, hsa-miR-339-3p, hsa-miR-339-5p, hsa-miR-33a, hsa-miR-33a*, hsa-miR-33b, hsa-miR-33b*, hsa-miR-340, hsa-miR-340*, hsa-miR-342-3p, hsa-miR-342-5p, hsa-miR-345, hsa-miR-346, hsa-miR-34a, hsa-miR-34a*, hsa-miR-34b, hsa-miR-34b*, hsa-miR-34c-3p, hsa-miR-34c-5p, hsa-miR-361-3p, hsa-miR-361-5p, hsa-miR-362-3p, hsa-miR-362-5p, hsa-miR-363, hsa-miR-363*, hsa-miR-365, hsa-miR-367, hsa-miR-367*, hsa-miR-369-3p, hsa-miR-369-5p, hsa-miR-370, hsa-miR-371-3p, hsa-miR-371-5p, hsa-miR-372, hsa-miR-373, hsa-miR-373*, hsa-miR-374a, hsa-miR-374a*, hsa-miR-374b, hsa-miR-374b*, hsa-miR-375, hsa-miR-376a, hsa-miR-376a*, hsa-miR-376b, hsa-miR-376c, hsa-miR-377, hsa-miR-377*, hsa-miR-378, hsa-miR-378*, hsa-miR-379, hsa-miR-379*, hsa-miR-380, hsa-miR-380*, hsa-miR-381, hsa-miR-382, hsa-miR-383, hsa-miR-384, hsa-miR-409-3p, hsa-miR-409-5p, hsa-miR-410, hsa-miR-411, hsa-miR-411*, hsa-miR-412, hsa-miR-421, hsa-miR-422a, hsa-miR-423-3p, hsa-miR-423-5p, hsa-miR-424, hsa-miR-424*, hsa-miR-425, hsa-miR-425*, hsa-miR-429, hsa-miR-431, hsa-miR-431*, hsa-miR-432, hsa-miR-432*, hsa-miR-433, hsa-miR-448, hsa-miR-449a, hsa-miR-449b, hsa-miR-450a, hsa-miR-450b-3p, hsa-miR-450b-5p, hsa-miR-451, hsa-miR-452, hsa-miR-452*, hsa-miR-453, hsa-miR-454, hsa-miR-454*, hsa-miR-455-3p, hsa-miR-455-5p, hsa-miR-483-3p, hsa-miR-483-5p, hsa-miR-484, hsa-miR-485-3p, hsa-miR-485-5p, hsa-miR-486-3p, hsa-miR-486-5p, hsa-miR-487a, hsa-miR-487b, hsa-miR-488, hsa-miR-488*, hsa-miR-489, hsa-miR-490-3p, hsa-miR-490-5p, hsa-miR-491-3p, hsa-miR-491-5p, hsa-miR-492, hsa-miR-493, hsa-miR-493*, hsa-miR-494, hsa-miR-495, hsa-miR-496, hsa-miR-497, hsa-miR-497*, hsa-miR-498, hsa-miR-499-3p, hsa-miR-499-5p, hsa-miR-500, hsa-miR-500*, hsa-miR-501-3p, hsa-miR-501-5p, hsa-miR-502-3p, hsa-miR-502-5p, hsa-miR-503, hsa-miR-504, hsa-miR-505, hsa-miR-505*, hsa-miR-506, hsa-miR-507, hsa-miR-508-3p, hsa-miR-508-5p, hsa-miR-509-3-5p, hsa-miR-509-3p, hsa-miR-509-5p, hsa-miR-510, hsa-miR-511, hsa-miR-512-3p, hsa-miR-512-5p, hsa-miR-513a-3p, hsa-miR-513a-5p, hsa-miR-513b, hsa-miR-513c, hsa-miR-514, hsa-miR-515-3p, hsa-miR-515-5p, hsa-miR-516a-3p, hsa-miR-516a-5p, hsa-miR-516b, hsa-miR-517*, hsa-miR-517a, hsa-miR-517b, hsa-miR-517c, hsa-miR-518a-3p, hsa-miR-518a-5p, hsa-miR-518b, hsa-miR-518c, hsa-miR-518c*, hsa-miR-518d-3p, hsa-miR-518d-5p, hsa-miR-518e, hsa-miR-518e*, hsa-miR-518f, hsa-miR-518f*, hsa-miR-519a, hsa-miR-519b-3p, hsa-miR-519c-3p, hsa-miR-519d, hsa-miR-519e, hsa-miR-519e*, hsa-miR-520a-3p, hsa-miR-520a-5p, hsa-miR-520b, hsa-miR-520c-3p, hsa-miR-520d-3p, hsa-miR-520d-5p, hsa-miR-520e, hsa-miR-520f, hsa-miR-520g, hsa-miR-520h, hsa-miR-521, hsa-miR-522, hsa-miR-523, hsa-miR-524-3p, hsa-miR-524-5p, hsa-miR-525-3p, hsa-miR-525-5p, hsa-miR-526b, hsa-miR-526b*, hsa-miR-532-3p, hsa-miR-532-5p, hsa-miR-539, hsa-miR-541, hsa-miR-541*, hsa-miR-542-3p, hsa-miR-542-5p, hsa-miR-543, hsa-miR-544, hsa-miR-545, hsa-miR-545*, hsa-miR-548a-3p, hsa-miR-548a-5p, hsa-miR-548b-3p, hsa-miR-548b-5p, hsa-miR-548c-3p, hsa-miR-548c-5p, hsa-miR-548d-3p, hsa-miR-548d-5p, hsa-miR-548e, hsa-miR-548f, hsa-miR-548g, hsa-miR-548h, hsa-miR-548i, hsa-miR-548j, hsa-miR-548k, hsa-miR-548l, hsa-miR-548m, hsa-miR-548n, hsa-miR-548o, hsa-miR-548p, hsa-miR-549, hsa-miR-550, hsa-miR-550*, hsa-miR-551a, hsa-miR-551b, hsa-miR-551b*, hsa-miR-552, hsa-miR-553, hsa-miR-554, hsa-miR-555, hsa-miR-556-3p, hsa-miR-556-5p, hsa-miR-557, hsa-miR-558, hsa-miR-559, hsa-miR-561, hsa-miR-562, hsa-miR-563, hsa-miR-564, hsa-miR-566, hsa-miR-567, hsa-miR-568, hsa-miR-569, hsa-miR-570, hsa-miR-571, hsa-miR-572, hsa-miR-573, hsa-miR-574-3p, hsa-miR-574-5p, hsa-miR-575, hsa-miR-576-3p, hsa-miR-576-5p, hsa-miR-577, hsa-miR-578, hsa-miR-579, hsa-miR-580, hsa-miR-581, hsa-miR-582-3p, hsa-miR-582-5p, hsa-miR-583, hsa-miR-584, hsa-miR-585, hsa-miR-586, hsa-miR-587, hsa-miR-588, hsa-miR-589, hsa-miR-589*, hsa-miR-590-3p, hsa-miR-590-5p, hsa-miR-591, hsa-miR-592, hsa-miR-593, hsa-miR-593*, hsa-miR-595, hsa-miR-596, hsa-miR-597, hsa-miR-598, hsa-miR-599, hsa-miR-600, hsa-miR-601, hsa-miR-602, hsa-miR-603, hsa-miR-604, hsa-miR-605, hsa-miR-606, hsa-miR-607, hsa-miR-608, hsa-miR-609, hsa-miR-610, hsa-miR-611, hsa-miR-612, hsa-miR-613, hsa-miR-614, hsa-miR-615-3p, hsa-miR-615-5p, hsa-miR-616, hsa-miR-616*, hsa-miR-617, hsa-miR-618, hsa-miR-619, hsa-miR-620, hsa-miR-621, hsa-miR-622, hsa-miR-623, hsa-miR-624, hsa-miR-624*, hsa-miR-625, hsa-miR-625*, hsa-miR-626, hsa-miR-627, hsa-miR-628-3p, hsa-miR-628-5p, hsa-miR-629, hsa-miR-629*, hsa-miR-630, hsa-miR-631, hsa-miR-632, hsa-miR-633, hsa-miR-634, hsa-miR-635, hsa-miR-636, hsa-miR-637, hsa-miR-638, hsa-miR-639, hsa-miR-640, hsa-miR-641, hsa-miR-642, hsa-miR-643, hsa-miR-644, hsa-miR-645, hsa-miR-646, hsa-miR-647, hsa-miR-648, hsa-miR-649, hsa-miR-650, hsa-miR-651, hsa-miR-652, hsa-miR-653, hsa-miR-654-3p, hsa-miR-654-5p, hsa-miR-655, hsa-miR-656, hsa-miR-657, hsa-miR-658, hsa-miR-659, hsa-miR-660, hsa-miR-661, hsa-miR-662, hsa-miR-663, hsa-miR-663b, hsa-miR-664, hsa-miR-664*, hsa-miR-665, hsa-miR-668, hsa-miR-671-3p, hsa-miR-671-5p, hsa-miR-675, hsa-miR-7, hsa-miR-708, hsa-miR-708*, hsa-miR-7-1*, hsa-miR-7-2*, hsa-miR-720, hsa-miR-744, hsa-miR-744*, hsa-miR-758, hsa-miR-760, hsa-miR-765, hsa-miR-766, hsa-miR-767-3p, hsa-miR-767-5p, hsa-miR-768-3p, hsa-miR-768-5p, hsa-miR-769-3p, hsa-miR-769-5p, hsa-miR-770-5p, hsa-miR-802, hsa-miR-873, hsa-miR-874, hsa-miR-875-3p, hsa-miR-875-5p, hsa-miR-876-3p, hsa-miR-876-5p, hsa-miR-877, hsa-miR-877*, hsa-miR-885-3p, hsa-miR-885-5p, hsa-miR-886-3p, hsa-miR-886-5p, hsa-miR-887, hsa-miR-888, hsa-miR-888*, hsa-miR-889, hsa-miR-890, hsa-miR-891a, hsa-miR-891b, hsa-miR-892a, hsa-miR-892b, hsa-miR-9, hsa-miR-9*, hsa-miR-920, hsa-miR-921, hsa-miR-922, hsa-miR-923, hsa-miR-924, hsa-miR-92a, hsa-miR-92a-1*, hsa-miR-92a-2*, hsa-miR-92b, hsa-miR-92b*, hsa-miR-93, hsa-miR-93*, hsa-miR-933, hsa-miR-934, hsa-miR-935, hsa-miR-936, hsa-miR-937, hsa-miR-938, hsa-miR-939, hsa-miR-940, hsa-miR-941, hsa-miR-942, hsa-miR-943, hsa-miR-944, hsa-miR-95, hsa-miR-96, hsa-miR-96*, hsa-miR-98, hsa-miR-99a, hsa-miR-99a*, hsa-miR-99b, and hsa-miR-99b*. In some embodiments, pri-miRNA sequences of the foregoing miRNAs may be useful as AmiRNA backbones.

In some embodiments, an isolated nucleic acid encodes one or more miR-375 molecules. Human miR-375 is encoded on human chromosome 2. The nucleic acid sequence of mature human miR-375 is set forth in Accession Number MIMAT0000728 (SEQ ID NO: 1). In some embodiments, the miR-375 molecule comprises the sequence set forth in SEQ ID NO: 1. In some embodiments, the pri-miR-375 is encoded by a nucleic acid comprising the sequence set forth in SEQ ID NO: 2. In some embodiments, the isolated nucleic acid comprises the pri-miR-375 sequence as set forth in SEQ ID NO: 2. In some embodiments, the isolated nucleic acid comprises an AmiRNA encoding one or more miR-375 molecules (e.g., 1, 2, 3, 4, 5, or more miR-375 molecules).

In some embodiments, the isolated nucleic acid comprises one or more inhibitory nucleic acid targeting one or more of the downstream targets of miR-375. In some embodiments, the inhibitory nucleic acid specifically binds to (e.g., hybridizes with) at least two (e.g., 2, 3, 4, 5, 6, 7, 8, 9, 10, 11, 12, 13, 14, 15, 16, 17, 18, 19, 20, 21, 22, 23, 24, 25, or more) continuous bases of a mRNA targeted by of miR-375 (e.g., CYB5b, SLC16A2, and/or ACSL5, etc.). As used herein “continuous bases” refers to two or more nucleotide bases that are covalently bound (e.g., by one or more phosphodiester bond, etc.) to each other (e.g., as part of a nucleic acid molecule). In some embodiments, the at least one inhibitory nucleic acid is at least about 50%, at least about 60%, at least about 70%, at least about 80%, at least about 90%, at least about 95%, at least about 99% or about 100% identical to the two or more (e.g., 2, 3, 4, 5, 6, 7, 8, 9, 10, 11, 12, 13, 14, 15, 16, 17, 18, 19, 20, 21, 22, 23, 24, 25, or more) continuous nucleotide bases of a mRNA targeted by of miR-375 (e.g., CYB5b, SLC16A2, ACSL5, etc.).

In some embodiments, the present disclosure provides an isolated nucleic acid comprising a transgene encoding an inhibitory nucleic acid targeting the human SLC16A2 gene (GeneID: 6567), which encodes the monocarboxylate transporter 8 protein. The monocarboxylate transporter 8 protein is an active transporter protein that transports a variety of iodo-thyronines. In some embodiments, the SLC16A2 gene is represented by the NCBI Accession Number NM_006517.5. In some embodiments, the monocarboxylate transporter 8 protein is represented by the NCBI Accession Number NP_006508.2. In some aspects, the disclosure relates to an isolated nucleic acid comprising a transgene encoding an inhibitory nucleic acid to reduce SLC16A2 expression (e.g., expression of one or more gene products from an SLC16A2 gene). In some embodiments, an isolated nucleic acid of the present disclosure comprises one or more inhibitory nucleic acid is a sequence comprising at least 6 (e.g., 6, 7, 8, 9, 10, 11, 12, 13, 14, 15, 16, 17, 18, 19, 20, 21, or more) contiguous nucleotides to the SLC16A2 gene represented by the NCBI Accession Number NM_006517.5. In some embodiments, the isolated nucleic acid encodes an inhibitory nucleic acid targeting the SLC16A2 gene, which comprises at least 6 (e.g., 6, 7, 8, 9, 10, 11, 12, 13, 14, 15, 16, 17, 18, 19, 20, 21, or more) contiguous nucleotides as set forth in SEQ ID NO: 3. In some embodiments, the isolated nucleic acid encodes an inhibitory nucleic acid targeting the SLC16A2 gene, which comprises at least 6 (e.g., 6, 7, 8, 9, 10, 11, 12, 13, 14, 15, 16, 17, 18, 19, 20, 21, or more) contiguous nucleotides as set forth in SEQ ID NO: 3, wherein the uracil nucleobases are replaced with thymine nucleobases. In some embodiments, the isolated nucleic acid encodes an inhibitory nucleic acid comprising SEQ ID NO: 3. In some embodiments, the isolated nucleic acid comprises an AmiRNA at least about 50%, at least about 60%, at least about 70%, at least about 80%, at least about 90%, at least about 95%, at least about 99% or about 100% identical to SEQ ID NO: 4. In some embodiments, the isolated nucleic acid comprises an AmiRNA as set forth in SEQ ID NO: 4.

In some embodiments, the present disclosure provides an isolated nucleic acid comprising a transgene encoding an inhibitory nucleic acid targeting the human ACSL5 gene (GeneID: 51703), which encodes the Long-chain-fatty-acid-CoA ligase 5 protein. The Long-chain-fatty-acid-CoA ligase 5 protein catalyzes the conversion of long-chain fatty acids to their active form acyl-CoAs for both synthesis of cellular lipids, and degradation via beta-oxidation. In some embodiments, the ACSL5 gene is represented by the NCBI Accession Number NM_016234.4, NM_203379.2, or NM_001387037.1. In some embodiments, the Long-chain-fatty-acid-CoA ligase 5 protein is represented by the NCBI Accession Number NP_057318.2. NP_976313.1 or NP_001373966.1. In some aspects, the disclosure relates to an isolated nucleic acid comprising a transgene encoding an inhibitory nucleic acid to reduce ACSL5 expression (e.g., expression of one or more gene products from an ACSL5 gene). In some embodiments, an isolated nucleic acid of the present disclosure comprises one or more inhibitory nucleic acid is a sequence comprising at least 6 (e.g., 6, 7, 8, 9, 10, 11, 12, 13, 14, 15, 16, 17, 18, 19, 20, 21, or more) contiguous nucleotides to the ACSL5 gene represented by the NCBI Accession Number NM_006517.5. In some embodiments, the isolated nucleic acid encodes an inhibitory nucleic acid targeting the ACSL5 gene, which comprises at least 6 (e.g., 6, 7, 8, 9, 10, 11, 12, 13, 14, 15, 16, 17, 18, 19, 20, 21, or more) contiguous nucleotides as set forth in SEQ ID NO: 5. In some embodiments, the isolated nucleic acid encodes an inhibitory nucleic acid targeting the ACSL5 gene, which comprises at least 6 (e.g., 6, 7, 8, 9, 10, 11, 12, 13, 14, 15, 16, 17, 18, 19, 20, 21, or more) contiguous nucleotides as set forth in SEQ ID NO: 5, wherein the uracil nucleobases are replaced with thymine nucleobases. In some embodiments, the isolated nucleic acid encodes an inhibitory nucleic acid comprising SEQ ID NO: 5. In some embodiments, the isolated nucleic acid comprises an AmiRNA at least about 50%, at least about 60%, at least about 70%, at least about 80%, at least about 90%, at least about 95%, at least about 99% or about 100% identical to SEQ ID NO: 6. In some embodiments, the isolated nucleic acid comprises an AmiRNA as set forth in SEQ ID NO: 6.

In some embodiments, the present disclosure provides an isolated nucleic acid comprising a transgene encoding an inhibitory nucleic acid (e.g., artificial microRNA) targeting the human CYB5b gene (GeneID: 80777), which encodes the cytochrome b5 type B protein. The cytochrome b5 type B protein is a membrane bound hemoprotein which function as an electron carrier for several membrane bound oxygenases. In some embodiments, the CYB5b gene is represented by the NCBI Accession Number NM_030579.3. In some embodiments, the cytochrome b5 type B protein is represented by the NCBI Accession Number NP_085056.2. In some aspects, the disclosure relates to an isolated nucleic acid comprising a transgene encoding an inhibitory nucleic acid to reduce CYB5b expression (e.g., expression of one or more gene products from an CYB5b gene). In some embodiments, an isolated nucleic acid of the present disclosure comprises one or more inhibitory nucleic acid is encoded by a sequence comprising at least 6 (e.g., 6, 7, 8, 9, 10, 11, 12, 13, 14, 15, 16, 17, 18, 19, 20, 21, or more) contiguous nucleotides to the CYB5b gene represented by the NCBI Accession Number NM_030579.3. In some embodiments, the isolated nucleic acid encodes an inhibitory nucleic acid targeting the CYB5b gene, which comprises at least 6 (e.g., 6, 7, 8, 9, 10, 11, 12, 13, 14, 15, 16, 17, 18, 19, 20, 21, or more) contiguous nucleotides as set forth in SEQ ID NO: 7. In some embodiments, the isolated nucleic acid encodes an inhibitory nucleic acid targeting the CYB5b gene, which comprises at least 6 (e.g., 6, 7, 8, 9, 10, 11, 12, 13, 14, 15, 16, 17, 18, 19, 20, 21, or more) contiguous nucleotides as set forth in SEQ ID NO: 7, wherein the uracil nucleobases are replaced with thymine nucleobases. In some embodiments, the isolated nucleic acid encodes an inhibitory nucleic acid comprising SEQ ID NO: 7. In some embodiments, the isolated nucleic acid comprises an AmiRNA at least about 50%, at least about 60%, at least about 70%, at least about 80%, at least about 90%, at least about 95%, at least about 99% or about 100% identical to SEQ ID NO: 8. In some embodiments, the isolated nucleic acid comprises an AmiRNA as set forth in SEQ ID NO: 8.

In some embodiment, an isolated nucleic acid comprises a transgene encoding miR-375 (e.g., SEQ ID NO: 1). In some embodiments, an isolated nucleic acid comprises a transgene encoding an inhibitory nucleic acid targeting ACSL5 (e.g., SEQ ID NO: 5). In some embodiments, an isolated nucleic acid comprises a transgene encoding an inhibitory nucleic acid targeting SLC16A2 (e.g., SEQ ID NO: 3). In some embodiments, an isolated nucleic acid comprises a transgene encoding an inhibitory nucleic acid targeting CYB5b (e.g., SEQ ID NO: 7). In some embodiment, an isolated nucleic acid comprises a transgene encoding miR-375 (e.g., SEQ ID NO: 1), and an inhibitory nucleic acid targeting ACSL5 (e.g., SEQ ID NO: 5). In some embodiment, an isolated nucleic acid comprises a transgene encoding miR-375 (e.g., SEQ ID NO: 1), and an inhibitory nucleic acid targeting SLC16A2 (e.g., SEQ ID NO: 3). In some embodiment, an isolated nucleic acid comprises a transgene encoding miR-375 (e.g., SEQ ID NO: 1), and an inhibitory nucleic acid targeting CYB5b (e.g., SEQ ID NO: 7). In some embodiment, an isolated nucleic acid comprises a transgene encoding ACSL5 (e.g., SEQ ID NO: 5), and an inhibitory nucleic acid targeting CYB5b (e.g., SEQ ID NO: 7). In some embodiment, an isolated nucleic acid comprises a transgene encoding SLC16A2 (e.g., SEQ ID NO: 3), and an inhibitory nucleic acid targeting CYB5b (e.g., SEQ ID NO: 7). In some embodiment, an isolated nucleic acid comprises a transgene encoding SLC16A2 (e.g., SEQ ID NO: 3), and an inhibitory nucleic acid targeting ACSL5 (e.g., SEQ ID NO: 5). In some embodiment, an isolated nucleic acid comprises a transgene encoding miR-375 (e.g., SEQ ID NO: 1), an inhibitory nucleic acid targeting ACSL5 (e.g., SEQ ID NO: 5), and an inhibitory nucleic acid targeting SLC16A2 (e.g., SEQ ID NO: 3). In some embodiment, an isolated nucleic acid comprises a transgene encoding miR-375 (e.g., SEQ ID NO: 1), an inhibitory nucleic acid targeting ACSL5 (e.g., SEQ ID NO: 5), and an inhibitory nucleic acid targeting CYB5b (e.g., SEQ ID NO: 7). In some embodiment, an isolated nucleic acid comprises a transgene encoding miR-375 (e.g., SEQ ID NO: 1), an inhibitory nucleic acid targeting SLC16A2 (e.g., SEQ ID NO: 3), and an inhibitory nucleic acid targeting CYB5b (e.g., SEQ ID NO: 7). In some embodiment, an isolated nucleic acid comprises a transgene encoding an inhibitory nucleic acid targeting SLC16A2 (e.g., SEQ ID NO: 3), an inhibitory nucleic acid targeting ACSL5 (e.g., SEQ ID NO: 5), and an inhibitory nucleic acid targeting CYB5b (e.g., SEQ ID NO: 7). In some embodiment, an isolated nucleic acid comprises a transgene encoding miR-375 (e.g., SEQ ID NO: 1), an inhibitory nucleic acid targeting SLC16A2 (e.g., SEQ ID NO: 3), an inhibitory nucleic acid targeting ACSL5 (e.g., SEQ ID NO: 5), and an inhibitory nucleic acid targeting CYB5b (e.g., SEQ ID NO: 7).

In some embodiment, an isolated nucleic acid comprises a pri-miRNA comprising the nucleic acid of SEQ ID NO: 2. In some embodiments, an isolated nucleic acid comprises an AmiRNA comprising the nucleic acid of SEQ ID NO: 6. In some embodiments, an isolated nucleic acid comprises an AmiRNA comprising the nucleic acid of SEQ ID NO: 4. In some embodiments, an isolated nucleic acid comprises an AmiRNA comprising the nucleic acid of SEQ ID NO: 8. In some embodiment, an isolated nucleic acid comprises a pri-miRNA comprising the nucleic acid of SEQ ID NO: 2, and an AmiRNA comprising the nucleic acid of SEQ ID NO: 6. In some embodiment, an isolated nucleic acid comprises a pri-miRNA comprising the nucleic acid of SEQ ID NO: 2, and an AmiRNA comprising the nucleic acid of SEQ ID NO: 4. In some embodiment, an isolated nucleic acid comprises a pri-miRNA comprising the nucleic acid of SEQ ID NO: 2, and an AmiRNA comprising the nucleic acid of SEQ ID NO: 8. In some embodiment, an isolated nucleic acid comprises an AmiRNA comprising the nucleic acid of SEQ ID NO: 6, and an AmiRNA comprising the nucleic acid of SEQ ID NO: 8. In some embodiment, an isolated nucleic acid comprises an AmiRNA comprising the nucleic acid of SEQ ID NO: 4, and an AmiRNA comprising the nucleic acid of SEQ ID NO: 8. In some embodiment, an isolated nucleic acid comprises an AmiRNA comprising the nucleic acid of SEQ ID NO: 4, and an AmiRNA comprising the nucleic acid of SEQ ID NO: 6. In some embodiment, an isolated nucleic acid comprises a a pri-miRNA comprising the nucleic acid of SEQ ID NO: 2, an AmiRNA comprising the nucleic acid of SEQ ID NO: 6, and an AmiRNA comprising the nucleic acid of SEQ ID NO: 4. In some embodiment, an isolated nucleic acid comprises a pri-miRNA comprising the nucleic acid of SEQ ID NO: 2, an AmiRNA comprising the nucleic acid of SEQ ID NO: 6, and an AmiRNA comprising the nucleic acid of SEQ ID NO: 8. In some embodiment, an isolated nucleic acid comprises a pri-miRNA comprising the nucleic acid of SEQ ID NO: 2, an AmiRNA comprising the nucleic acid of SEQ ID NO: 4, and an AmiRNA comprising the nucleic acid of SEQ ID NO: 8. In some embodiment, an isolated nucleic acid comprises an AmiRNA comprising the nucleic acid of SEQ ID NO: 4, an AmiRNA comprising the nucleic acid of SEQ ID NO: 6, and an AmiRNA comprising the nucleic acid of SEQ ID NO: 8. In some embodiment, an isolated nucleic acid comprises a pri-miRNA comprising the nucleic acid of SEQ ID NO: 2, an AmiRNA comprising the nucleic acid of SEQ ID NO: 4, an AmiRNA comprising the nucleic acid of SEQ ID NO: 6, and an AmiRNA comprising the nucleic acid of SEQ ID NO: 8.

In some embodiments, an inhibitory nucleic acid as described by the disclosure comprises a sequence in which thymine nucleobases have been replaced with uracil nucleobases. In some embodiments, an inhibitory nucleic acid comprises a sequence in which every thymine nucleobases has been replaced with a uracil nucleobase. In some embodiments, an inhibitory nucleic acid comprises a sequence in which 1, 2, 3, 4, 5, 6, 7, 8, 9, 10, 11, 12, 13, 14, 15, or more thymine nucleobases have been replaced with uracil nucleobases.

In some embodiments, an isolated nucleic acid described herein comprises one or more inhibitory nucleic acids that decrease expression and/or activity of a target gene (e.g., CYB5b, SLC16A2, and/or ACSL5) by between 50% and 99% (e.g., any integer between 50% and 99%, inclusive). In some embodiments, an isolated nucleic acid described herein comprises one or more inhibitory nucleic acids that decrease expression of a target gene (e.g., CYB5b, SLC16A2, and/or ACSL5) by between 75% and 90%. In some aspects, an isolated nucleic acid described herein comprises one or more inhibitory nucleic acids that decrease expression of a target gene (e.g., CYB5b, SLC16A2, and/or ACSL5) by between 80% and 99%. In some embodiments, an isolated nucleic acid described herein comprises one or more inhibitory nucleic acids that decrease expression of CYB5b, SLC16A2, and/or ACSL5 gene by between 50% and 99% (e.g., any integer between 50% and 99%, inclusive). In some embodiments, an isolated nucleic acid described herein comprises one or more inhibitory nucleic acids that decrease expression of CYB5b, SLC16A2, and/or ACSL5 gene by between 75% and 90%. In some aspects, an isolated nucleic acid described herein comprises one or more inhibitory nucleic acids that decrease expression of CYB5b, SLC16A2, and/or ACSL5 gene by between 80% and 99%.

In some embodiments, an isolated comprises encoding various inhibitory nucleic acids as described herein is a multicistronic cassette. In some embodiments, a multicistronic expression construct comprises two or more expression cassettes encoding one or more inhibitory nucleic acid (e.g., miR-375, inhibitory nucleic acids targeting CYB5b, SLC16A2, and/or ACSL5, or a combination thereof) described herein.

In some embodiments, multicistronic expression constructs are comprise expression cassettes that are positioned in different ways. For example, in some embodiments, a multicistronic expression construct is provided in which a first expression cassette (e.g., an expression cassette encoding miR-375) is positioned adjacent to a second expression cassette (e.g., an expression cassette encoding an inhibitory nucleic acid targeting CYB5b, SLC16A2, or ACSL5). In some embodiments, a multicistronic expression construct is provided in which a first expression cassette comprises an intron, and a second expression cassette is positioned within the intron of the first expression cassette. In some embodiments, the second expression cassette, positioned within an intron of the first expression cassette, comprises a promoter and a nucleic acid sequence encoding a gene product operatively linked to the promoter.

In different embodiments, multicistronic expression constructs are provided in which the expression cassettes are oriented in different ways. For example, in some embodiments, a multicistronic expression construct is provided in which a first expression cassette is in the same orientation as a second expression cassette. In some embodiments, a multicistronic expression construct is provided comprising a first and a second expression cassette in opposite orientations.

The term “orientation” as used herein in connection with expression cassettes, refers to the directional characteristic of a given cassette or structure. In some embodiments, an expression cassette harbors a promoter 5′ of the encoding nucleic acid sequence, and transcription of the encoding nucleic acid sequence runs from the 5′ terminus to the 3′ terminus of the sense strand, making it a directional cassette (e.g. 5′-promoter/(intron)/encoding sequence-3′). Since virtually all expression cassettes are directional in this sense, those of skill in the art can easily determine the orientation of a given expression cassette in relation to a second nucleic acid structure, for example, a second expression cassette, a viral genome, or, if the cassette is comprised in an AAV construct, in relation to an AAV ITR.

For example, if a given nucleic acid construct comprises two expression cassettes in the configuration 5′-promoter 1/encoding sequence 1—promoter2/encoding sequence 2-3′,

>>>>>>>>>>>>>>>>>>>>>>> >>>>>>>>>>>>>>>>>>>>>>>

the expression cassettes are in the same orientation, the arrows indicate the direction of transcription of each of the cassettes. For another example, if a given nucleic acid construct comprises a sense strand comprising two expression cassettes in the configuration 5′-promoter 1/encoding sequence 1—encoding sequence 2/promoter 2-3′,

>>>>>>>>>>>>>>>>>>>>>>> <<<<<<<<<<<<<<<<<<<<<

the expression cassettes are in opposite orientation to each other and, as indicated by the arrows, the direction of transcription of the expression cassettes, are opposed. In this example, the strand shown comprises the antisense strand of promoter 2 and encoding sequence 2.

For another example, if an expression cassette is comprised in an AAV construct, the cassette can either be in the same orientation as an AAV ITR, or in opposite orientation. AAV ITRs are directional. For example, the 3′ITR would be in the same orientation as the promoter1/encoding sequence 1 expression cassette of the examples above, but in opposite orientation to the 5′ITR, if both ITRs and the expression cassette would be on the same nucleic acid strand.

A large body of evidence suggests that multicistronic expression constructs often do not achieve optimal expression levels as compared to expression systems containing only one cistron. One of the suggested causes of sub-par expression levels achieved with multicistronic expression constructs comprising two or more promoter elements is the phenomenon of promoter interference (see, e.g., Curtin J A, Dane A P, Swanson A, Alexander I E, Ginn S L. Bidirectional promoter interference between two widely used internal heterologous promoters in a late-generation lentiviral construct. Gene Ther. 2008 March; 15(5):384-90; and Martin-Duque P, Jezzard S, Kaftansis L, Vassaux G. Direct comparison of the insulating properties of two genetic elements in an adenoviral vector containing two different expression cassettes. Hum Gene Ther. 2004 October; 15(10):995-1002; both references incorporated herein by reference for disclosure of promoter interference phenomenon). Various strategies have been suggested to overcome the problem of promoter interference, for example, by producing multicistronic expression constructs comprising only one promoter driving transcription of multiple encoding nucleic acid sequences separated by internal ribosomal entry sites, or by separating cistrons comprising their own promoter with transcriptional insulator elements. All suggested strategies to overcome promoter interference are burdened with their own set of problems, though. For example, single-promoter driven expression of multiple cistrons usually results in uneven expression levels of the cistrons. Further some promoters cannot efficiently be isolated and isolation elements are not compatible with some gene transfer vectors, for example, some retroviral vectors.

In some embodiments, a multicistronic expression construct is provided that allows efficient expression of a first encoding nucleic acid sequence driven by a first promoter and of a second encoding nucleic acid sequence driven by a second promoter without the use of transcriptional insulator elements. Various configurations of such multicistronic expression constructs are provided herein, for example, expression constructs harboring a first expression cassette comprising an intron and a second expression cassette positioned within the intron, in either the same or opposite orientation as the first cassette. Other configurations are described in more detail elsewhere herein.

In some embodiments, multicistronic expression constructs are provided allowing for efficient expression of two or more encoding nucleic acid sequences. In some embodiments, the multicistronic expression construct comprises two expression cassettes. In some embodiments, a first expression cassette of a multicistronic expression construct as provided herein comprises a first RNA polymerase II promoter and a second expression cassette comprises a second RNA polymerase II promoter. In some embodiments, a first expression cassette of a multicistronic expression construct as provided herein comprises an RNA polymerase II promoter and a second expression cassette comprises an RNA polymerase III promoter.

In some embodiments, the isolated nucleic acid comprises inverted terminal repeats. The isolated nucleic acids of the disclosure may be recombinant adeno-associated virus (AAV) vectors (rAAV vectors). In some embodiments, an isolated nucleic acid as described by the disclosure comprises a region (e.g., a first region) comprising a first adeno-associated virus (AAV) inverted terminal repeat (ITR), or a variant thereof. The isolated nucleic acid (e.g., the recombinant AAV vector) may be packaged into a capsid protein and administered to a subject and/or delivered to a selected target cell. “Recombinant AAV (rAAV) vectors” are typically composed of, at a minimum, a transgene and its regulatory sequences, and 5′ and 3′ AAV inverted terminal repeats (ITRs).

Generally, ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al., “Molecular Cloning. A Laboratory Manual”, 2d ed., Cold Spring Harbor Laboratory, New York (1989); and K. Fisher et al., J Virol., 70:520 532 (1996)). An example of such a molecule employed in the disclosure is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types. In some embodiments, the isolated nucleic acid further comprises a region (e.g., a second region, a third region, a fourth region, etc.) comprising a second AAV ITR. In some embodiments, an isolated nucleic acid encoding a transgene is flanked by AAV ITRs (e.g., in the orientation 5′-ITR-transgene-ITR-3′). In some embodiments, the AAV ITRs are selected from the group consisting of AAV1 ITR, AAV2 ITR, AAV3 ITR, AAV4 ITR, AAV5 ITR, and AAV6 ITR. In some embodiments, the second ITR is a mutant ITR that lacks a functional terminal resolution site (TRS). The term “lacking a terminal resolution site” can refer to an AAV ITR that comprises a mutation (e.g., a sense mutation such as a non-synonymous mutation, or missense mutation) that abrogates the function of the terminal resolution site (TRS) of the ITR, or to a truncated AAV ITR that lacks a nucleic acid sequence encoding a functional TRS (e.g., a ΔTRS ITR, or AITR). Without wishing to be bound by any particular theory, a rAAV vector comprising an ITR lacking a functional TRS produces a self-complementary rAAV vector, for example as described by McCarthy (2008) Molecular Therapy 16(10):1648-1656. In some embodiments, vectors described herein comprise one or more AAV ITRs, and at least one ITR is an ITR variant of a known AAV serotype ITR. In some embodiments, the AAV ITR variant is a synthetic AAV ITR (e.g., AAV ITRs that do not occur naturally). In some embodiments, the AAV ITR variant is a hybrid ITR (e.g., a hybrid ITR comprises sequences derived from ITRs of two or more different AAV serotypes).

In addition to the major elements identified above for the recombinant AAV vector, the vector also includes conventional control elements which are operably linked with elements of the transgene in a manner that permits its transcription, translation and/or expression in a cell transfected with the vector or infected with the virus produced by the disclosure. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (e.g., Kozak consensus sequence); sequences that enhance protein stability; and when desired, sequences that enhance secretion of the encoded product. A number of expression control sequences, including promoters which are native, constitutive, inducible and/or tissue-specific, are known in the art and may be utilized.

As used herein, a nucleic acid sequence (e.g., coding sequence) and regulatory sequences are said to be operably linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly two or more coding regions are operably linked when they are linked in such a way that their transcription from a common promoter results in the expression of two or more proteins having been translated in frame. In some embodiments, operably linked coding sequences yield a fusion protein.

It should be appreciated that in cases where a transgene encodes more than one inhibitory nucleic acids, each coding sequence may be positioned in any suitable location within the transgene. For example, a nucleic acid encoding a first inhibitory nucleic acid may be positioned downstream or upstream of a second inhibitory nucleic acid.

A “promoter” refers to a DNA sequence recognized by the synthetic machinery of the cell, or introduced synthetic machinery, required to initiate the specific transcription of a gene. The phrases “operatively linked,” “operatively positioned,” “under control” or “under transcriptional control” means that the promoter is in the correct location and orientation in relation to the nucleic acid to control RNA polymerase initiation and expression of the gene.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al., Cell, 41:521-530 (1985)], the SV40 promoter, the dihydrofolate reductase promoter, the β-actin promoter, the phosphoglycerol kinase (PGK) promoter, and the EF1α promoter [Invitrogen]. In some embodiments, a promoter is an RNA pol II promoter. In some embodiments, a promoter is an RNA pol III promoter, such as U6 or H1. In some embodiments, a promoter is an RNA pol II promoter. In some embodiments, a promoter is a chicken β-actin (CBA) promoter.

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In another embodiment, the native promoter for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: a liver-specific thyroxin binding globulin (TBG) promoter (see, e.g., Yan et al., Human thyroxine binding globulin (TBG) promoter directs efficient and sustaining transgene expression in liver-specific pattern, Gene. 2012 Sep. 15; 506(2):289-94)., an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a a-myosin heavy chain (a-MHC) promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor α-chain promoter, among others which will be apparent to the skilled artisan.

In some embodiments, a transgene which encodes at least one inhibitory nucleic acid is operably linked to a promoter. In some embodiments, the transgene encoding at least one inhibitory nucleic acid is and the transgene which encodes a selectable marker or reporter protein are operably linked to different promoters. In some embodiments, the promoter is a constitutive promoter. In some embodiments, the promoter is an inducible promoter. In some embodiments, the promoter is a tissue-specific promoter. In some embodiments, the tissue-specific promoter is a liver-specific promoter. In some embodiments, the liver-specific promoter is a TGB promoter.

Aspects of the disclosure relate to an isolated nucleic acid comprising more than one promoter (e.g., 2, 3, 4, 5, or more promoters). For example, in the context of a construct having a transgene comprising a first region (e.g., a first inhibitory nucleic acid) and an second region (e.g., a second inhibitory nucleic acid or a protein coding sequence, etc.) it may be desirable to drive expression of the first inhibitory nucleic acid coding region using a first promoter sequence (e.g., a first promoter sequence operably linked to the first region), and to drive expression of the second region with a second promoter sequence (e.g., a second promoter sequence operably linked to the second region). Generally, the first promoter sequence and the second promoter sequence can be the same promoter sequence or different promoter sequences. In some embodiments, the first promoter sequence (e.g., the promoter driving expression of the first region) is a RNA polymerase III (pol III) promoter sequence. Non-limiting examples of pol III promoter sequences include U6 and H1 promoter sequences. In some embodiments, the second promoter sequence (e.g., the promoter sequence driving expression of the second region) is a RNA polymerase II (pol II) promoter sequence. Non-limiting examples of pol II promoter sequences include T7, T3, SP6, RSV, and cytomegalovirus promoter sequences. In some embodiments, a pol III promoter sequence drives expression of the first region. In some embodiments, a pol II promoter sequence drives expression of the second region.

In some embodiments, the isolated nucleic acid may further comprise an expression cassette for a protein. For nucleic acids encoding proteins, a polyadenylation sequence generally is inserted following the transgene sequences and before the 3′ AAV ITR sequence. A rAAV construct useful in the present disclosure may also contain an intron, desirably located between the promoter/enhancer sequence and the transgene. One possible intron sequence is derived from SV-40, and is referred to as the SV-40 T intron sequence. Another vector element that may be used is an internal ribosome entry site (IRES). An IRES sequence is used to produce more than one polypeptide from a single gene transcript. An IRES sequence would be used to produce a protein that contain more than one polypeptide chains. Selection of these and other common vector elements are conventional and many such sequences are available [see, e.g., Sambrook et al., and references cited therein at, for example, pages 3.18 3.26 and 16.17 16.27 and Ausubel et al., Current Protocols in Molecular Biology, John Wiley & Sons, New York, 1989]. In some embodiments, a Foot and Mouth Disease Virus 2A sequence is included in polyprotein; this is a small peptide (approximately 18 amino acids in length) that has been shown to mediate the cleavage of polyproteins (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459). The cleavage activity of the 2A sequence has previously been demonstrated in artificial systems including plasmids and gene therapy vectors (AAV and retroviruses) (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459; de Felipe, P et al., Gene Therapy, 1999; 6: 198-208; de Felipe, Petal., Human Gene Therapy, 2000; 11: 1921-1931; and Klump, H et al., Gene Therapy, 2001; 8: 811-817).

In some embodiments, a vector described herein comprises a nucleic a nucleic acid sequence at least 70%, at least 80%, at least 85%, at least 90%, at least 95%, at least 96%, at least 97%, at least 98%, at least 99%, or 100% identical to nucleic acid sequence of any one of SEQ ID NOs: 9-12.

Recombinant Adeno-Associated Viruses (rAAVs)

In some aspects, the disclosure provides isolated adeno-associated viruses (AAVs). As used herein with respect to AAVs, the term “isolated” refers to an AAV that has been artificially produced or obtained. Isolated AAVs may be produced using recombinant methods. Such AAVs are referred to herein as “recombinant AAVs”. Recombinant AAVs (rAAVs) preferably have tissue-specific targeting capabilities, such that a nuclease and/or transgene of the rAAV will be delivered specifically to one or more predetermined tissue(s). The AAV capsid is an important element in determining these tissue-specific targeting capabilities. Thus, an rAAV having a capsid appropriate for the tissue being targeted can be selected.

Methods for obtaining recombinant AAVs having a desired capsid protein are well known in the art. (See, for example, US 2003/0138772), the contents of which are incorporated herein by reference in their entirety). Typically the methods involve culturing a host cell which contains a nucleic acid sequence encoding an AAV capsid protein; a functional rep gene; a recombinant AAV vector composed of AAV inverted terminal repeats (ITRs) and a transgene; and sufficient helper functions to permit packaging of the recombinant AAV vector into the AAV capsid proteins. In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, the functions of the capsid proteins are to protect the viral genome, deliver the genome and interact with the host. In some aspects, capsid proteins deliver the viral genome to a host in a tissue specific manner.

In some embodiments, an AAV capsid protein is of an AAV serotype selected from the group consisting of AAV1, AAV2, AAV3, AAV4, AAV5, AAV6, AAV7, AAV8, AAVrh8, AAV9, AAV10, AAVrh10, and AAV.PHP.B. In some embodiments, an AAV capsid protein is of a serotype derived from a non-human primate, for example AAVrh8 serotype. In some embodiments, an AAV capsid protein is of a serotype derived for broad and efficient liver transduction. In some embodiments, the capsid protein is of AAV serotype 8.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

In some embodiments, the instant disclosure relates to a host cell containing a nucleic acid that comprises a coding sequence encoding a transgene (e.g., a DNA binding domain fused to a transcriptional regulator domain). In some embodiments, the host cell is a mammalian cell, a yeast cell, a bacterial cell, an insect cell, a plant cell, or a fungal cell.

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al., J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with an AAV vector (comprising a transgene flanked by ITR elements) to be packaged into AAV particles, an AAV helper function vector, and an accessory function vector. An AAV helper function vector encodes the “AAV helper function” sequences (e.g., rep and cap), which function in trans for productive AAV replication and encapsidation. Preferably, the AAV helper function vector supports efficient AAV vector production without generating any detectable wild-type AAV virions (e.g., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (e.g., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpes virus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. In some embodiments, a host cell is a hepatocyte. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. In some embodiments, a vector is a viral vector, such as an rAAV vector, a lentiviral vector, an adenoviral vector, a retroviral vector, etc. Thus, the term includes cloning and expression vehicles, as well as viral vectors. In some embodiments, useful vectors are contemplated to be those vectors in which the nucleic acid segment to be transcribed is positioned under the transcriptional control of a promoter.

A “promoter” refers to a DNA sequence recognized by the synthetic machinery of the cell, or introduced synthetic machinery, required to initiate the specific transcription of a gene. The phrases “operatively linked”, “operatively positioned,” “under control” or “under transcriptional control” means that the promoter is in the correct location and orientation in relation to the nucleic acid to control RNA polymerase initiation and expression of the gene. The term “expression vector or construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product from a transcribed gene. The foregoing methods for packaging recombinant vectors in desired AAV capsids to produce the rAAVs of the disclosure are not meant to be limiting and other suitable methods will be apparent to the skilled artisan.

### Methods of Treating Acute Liver Failure (ALF)

Methods for inhibiting cytochrome p450-mediated toxicity in a cell or subject are provided by the disclosure. The methods typically involve administering to a cell or a subject an isolated nucleic acid or rAAV comprising a transgene which encodes at least one inhibitory nucleic acid that is miR-375 molecule or target one or more downstream targets of miR-375 (e.g., CYB5b, SLC16A2, ACSL5). In some embodiments, an inhibitory nucleic acid is an amiRNA, an shRNA, an siRNA, microRNA, or an antisense oligonucleotide (ASO).

In some aspects, the disclosure provides methods of modulating (e.g., increasing, decreasing, etc.) expression of a target gene in a cell. In some embodiments, the modulating is decreasing expression of a target gene (e.g., CYB5b, SLC16A2, ACSL5) in a cell. In some embodiments, a cell is a mammalian cell, such as a human cell, non-human primate cell, cat cell, mouse cell, dog cell, rat cell, hamster cell, etc. In some embodiments, a cell is a hepatocyte. In some embodiments, a cell is in a subject (e.g., in vivo).

In some embodiments, the disclosure provides methods of treating acute liver failure (ALF) (e.g., APAP overdose-induced ALF).

Administering an isolated nucleic acid or an rAAV encoding a transgene as described by the disclosure to a cell or subject, in some embodiments, results in decreased expression of a target gene (e.g., CYB5b, SLC16A2, ACSL5), which are downstream targets of miR-375. Thus, in some embodiments, compositions and methods described by the disclosure are useful for treating miR-375 associated diseases (e.g., ALF induced by APAP overdose).

In some embodiments, the subject has or is suspected of having ALF (e.g., APAP overdose induced ALF). A subject that “has or is suspected of having ALF” refers to a subject characterized by 1) one or more signs or symptoms of ALF, for example, yellowing of your skin and eyeballs (jaundice). pain in your upper right abdomen, abdominal swelling (ascites), nausea, vomiting, a general sense of feeling unwell (malaise), disorientation or confusion, sleepiness, etc., and/or 2) one or more abnormal laboratory results (e.g., increased Alanine transaminase (ALT)). In some embodiments, a subject has a history of APAP overdose. In some embodiments, the subject is characterized by aberrant (e.g., increased or decreased, relative to a healthy, normal cell or subject) level of N-acetyl-p-benzoquinone imine (NAPQI) adducts, which is a toxic byproduct of APAP. In some embodiments, administration of the isolated nucleic acid, the rAAV, or a pharmaceutical composition comprising the same result in a decreased level of NAPQI or ALT. In some embodiments, “decreased” level or activity is measured relative to expression or activity (e.g., by at least 10%, at least 20%, at least 30%, at least 40%, at least 50%, at least 60%, at least 70%, at least 80%, at least 90% or 100%) of that marker in a cell or subject who has not been administered one or more isolated nucleic acids, rAAVs, or compositions as described herein. In some embodiments, “decreased” expression or activity is measured relative to expression or activity of that transgene in the subject after the subject has been administered (e.g., gene expression is measured pre- and post-administration of) one or more isolated nucleic acids, rAAVs, or compositions as described herein. Methods of measuring gene expression or protein levels are known in the art and include, for example, quantitative PCR (qPCR), Western Blot, mass spectrometry (MS) assays, etc.

In some embodiments, administration of an isolated nucleic acid, rAAV, or composition as described by the disclosure results in an increase of miR-375 expression and/or activity, or a reduction of miR-375 target gene (e.g., CYB5b, SLC16A2, ACSL5) expression and/or activity in a subject between 2-fold and 100-fold (e.g., 2-fold, 5-fold, 10-fold, 50-fold, 100-fold, etc.) relative to miR-375 or miR-375 target gene expression and/or activity of a subject who has not been administered one or more compositions described by the disclosure.

The isolated nucleic acids, rAAVs, and compositions of the disclosure may be delivered to a subject in compositions according to any appropriate methods known in the art. For example, an rAAV, preferably suspended in a physiologically compatible carrier (e.g., in a composition), may be administered to a subject, i.e. host animal, such as a human, mouse, rat, cat, dog, sheep, rabbit, horse, cow, goat, pig, guinea pig, hamster, chicken, turkey, or a non-human primate (e.g., Macaque). In some embodiments a host animal does not include a human.

Delivery of the rAAVs to a mammalian subject may be by, for example, intramuscular injection or by administration into the bloodstream of the mammalian subject. Administration into the bloodstream may be by injection into a vein, an artery, or any other vascular conduit. In some embodiments, the rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue. Moreover, in certain instances, it may be desirable to deliver the virions to the liver of a subject. Recombinant AAVs may be delivered directly to the liver by injection into, e.g., the portal vein. In some embodiments, an rAAV as described in the disclosure are administered by intravenous injection. In some embodiments, rAAVs are administered by intrathecal injection. In some embodiments, rAAVs are delivered by intramuscular injection.

Aspects of the instant disclosure relate to compositions comprising a recombinant AAV comprising a capsid protein and a nucleic acid encoding a transgene, wherein the transgene comprises a nucleic acid sequence encoding one or more proteins. In some embodiments, the nucleic acid further comprises AAV ITRs. In some embodiments, a composition further comprises a pharmaceutically acceptable carrier.

The compositions of the disclosure may comprise an rAAV alone, or in combination with one or more other viruses (e.g., a second rAAV encoding having one or more different transgenes). In some embodiments, a composition comprises 1, 2, 3, 4, 5, 6, 7, 8, 9, 10, or more different rAAVs each having one or more different transgenes.

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions of the disclosure may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, parachlorophenol, and poloxamers (non-ionic surfactants) such as Pluronic® F-68. Suitable chemical stabilizers include gelatin and albumin.

The rAAVs are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., intraportal delivery to the liver), oral, inhalation (including intranasal and intratracheal delivery), intraocular, intravenous, intramuscular, subcutaneous, intradermal, intratumoral, and other parental routes of administration. Routes of administration may be combined, if desired.

The dose of rAAV virions required to achieve a particular “therapeutic effect,” e.g., the units of dose in genome copies/per kilogram of body weight (GC/kg), will vary based on several factors including, but not limited to: the route of rAAV virion administration, the level of gene or RNA expression required to achieve a therapeutic effect, the specific disease or disorder being treated, and the stability of the gene or RNA product. One of skill in the art can readily determine a rAAV virion dose range to treat a patient having a particular disease or disorder based on the aforementioned factors, as well as other factors that are well known in the art.

An effective amount of an rAAV is an amount sufficient to target infect an animal, target a desired tissue. In some embodiments, an effective amount of an rAAV is administered to the subject having ALF.

In some embodiments, rAAV compositions are formulated to reduce aggregation of AAV particles in the composition, particularly where high rAAV concentrations are present (e.g., ˜10¹³ GC/mL or more). Methods for reducing aggregation of rAAVs are well known in the art and, include, for example, addition of surfactants, pH adjustment, salt concentration adjustment, etc. (See, e.g., Wright F R, et al., Molecular Therapy (2005) 12, 171-178, the contents of which are incorporated herein by reference.)

Formulation of pharmaceutically-acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the rAAV-based therapeutic constructs in suitably formulated pharmaceutical compositions disclosed herein either intraportally, subcutaneously, intraopancreatically, intranasally, parenterally, intravenously, intramuscularly, intrathecally, or orally, intraperitoneally, or by inhalation. In some embodiments, the administration modalities as described in U.S. Pat. Nos. 5,543,158; 5,641,515 and 5,399,363 (each specifically incorporated herein by reference in its entirety) may be used to deliver rAAVs. In some embodiments, a preferred mode of administration is by portal vein injection.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, the term “treating” refers to the application or administration of a composition comprising an inhibitory nucleic acid (e.g., miR-375 or inhibitory nucleic acids targeting miR-375 targets) to a subject, who has ALF, with the purpose to cure, heal, alleviate, relieve, alter, remedy, ameliorate, improve, or affect the disorder, the symptom of the disease, or the predisposition toward ALF. In some embodiments, administration of the composition described herein results in an increased miR-375 activity by 2%, 5%, 10%, 20%, 30%, 40%, 50%, 60%, 70%, 80%, 90%, or 100% compared to a reference value. Methods of measuring miR-375 are known in the art. Non-limiting exemplary reference value can be miR-375 expression and/or activity of the same subject prior to receiving the treatment. In some embodiments, administration of a composition described herein results in a reduction of ALT by 2%, 5%, 10%, 20%, 30%, 40%, 50%, 60%, 70%, 80%, 90%, or 100% compared to a reference value. Methods of measuring ALT are known in the art. Non-limiting exemplary reference value can be level of ALT of the same subject prior to receiving the treatment. In some embodiments, administration of a composition described herein results in a reduction of NAPQI by 2%, 5%, 10%, 20%, 30%, 40%, 50%, 60%, 70%, 80%, 90%, or 100% compared to a reference value. Methods of measuring NAPQI are known in the art. Non-limiting exemplary reference value can be level of NAPQI of the same subject prior to receiving the treatment.

Alleviating ALF includes delaying the development or progression of the disease or reducing disease severity. Alleviating the disease does not necessarily require curative results. As used therein, “delaying” the development of a disease (e.g., ALF) means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result. “Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a disease (e.g., ALF).

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the rAAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

### Kits and Related Compositions

The agents described herein may, in some embodiments, be assembled into pharmaceutical or diagnostic or research kits to facilitate their use in therapeutic, diagnostic or research applications. A kit may include one or more containers housing the components of the disclosure and instructions for use. Specifically, such kits may include one or more agents described herein, along with instructions describing the intended application and the proper use of these agents. In certain embodiments agents in a kit may be in a pharmaceutical formulation and dosage suitable for a particular application and for a method of administration of the agents. Kits for research purposes may contain the components in appropriate concentrations or quantities for running various experiments.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflect approval by the agency of manufacture, use or sale for animal administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be in the form of a liquid, gel or solid (powder). The agents may be prepared sterilely, packaged in syringe and shipped refrigerated. Alternatively it may be housed in a vial or other container for storage. A second container may have other agents prepared sterilely. Alternatively the kit may include the active agents premixed and shipped in a syringe, vial, tube, or other container. The kit may have one or more or all of the components required to administer the agents to an animal, such as a syringe, topical application devices, or iv needle tubing and bag, particularly in the case of the kits for producing specific somatic animal models.

The kit may have a variety of forms, such as a blister pouch, a shrink wrapped pouch, a vacuum sealable pouch, a sealable thermoformed tray, or a similar pouch or tray form, with the accessories loosely packed within the pouch, one or more tubes, containers, a box or a bag. The kit may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kit may also include other components, depending on the specific application, for example, containers, cell media, salts, buffers, reagents, syringes, needles, a fabric, such as gauze, for applying or removing a disinfecting agent, disposable gloves, a support for the agents prior to administration etc.

The instructions included within the kit may involve methods for constructing an AAV vector as described herein. In addition, kits of the disclosure may include, instructions, a negative and/or positive control, containers, diluents and buffers for the sample, sample preparation tubes and a printed or electronic table of reference AAV sequence for sequence comparisons.

### EXAMPLES

### Example 1

Acetaminophen (APAP) overdose is the leading cause of acute liver failure (ALF) in many countries, including the United States. The high incidence of mortality associated with APAP-ALF can be attributed to its rapid onset and lack of an effective treatment. This example describes a preclinical study using APAP-ALF mouse model. Highly liver-tropic AAV8 was used to produce an rAAV for TBG promoter-driven, hepatocyte-specific, overexpression of miR-375, miR-122, or miR-125b. Following APAP challenge (350 mg/kg) and analyses of liver function (ALT), histology (H&E staining), mitochondrial function (the Seahorse assay) and transcriptome (RNA-seq), it was observed that miR-375 completely blocked ALF. This protection was observed in the APAP overdose ALF model but not in a Fas ligand-induced ALF model.

Mechanistically, RNA-seq data from liver transduced with AAV8-miR-375 indicates that genes regulated by miR-375 are enriched in the cytochrome P450 system (CYP), APAP-metabolizing enzymes responsible for cytotoxic byproduct NAPQI (N-acetyl-p-benzoquinone imine) formation, as well as NAPQI-scavenging Glutathione-S-transferases (GST) and Sulfotransferases (SULT). The significantly decreased CYP2E1 and increased GSTM1 expression were independently validated by RT-qPCR and Western blotting. Consistently, LC/MS-MS measurement revealed increased levels of liver glutathione (GSH) in the AAV-miR-375 treated mice before APAP dosing while NAPQI-protein adducts was significantly decreased after APAP challenge in miR-375 expressing liver. More strikingly, the overall APAP uptake in AAV-miR-375 transduced liver was significantly attenuated, indicating that miR-375 regulates active APAP transportation pathway.

Furthermore, while AAV-amiRs-mediated in vivo silencing of each potential miR-375 target genes, Slc16a2, Cyb5b and Acsl5, partially mimics miR-375-mediated protection, complete recapitulation of the protective effects from miR-375 was achieved in the combined administration of these three AAV-amiR vectors.

### Example 2

This example describes hepatocyte-specific overexpression of certain miRNAs (e.g., miR-375, etc.) blocks acute liver failure (ALF) in mice that have been administered an overdose (e.g., 350 mg/kg) of acetaminophen (APAP).

Several rAAVs were produced, including an rAAV encoding a miR-375 transgene under the control of a thyroxine binding globulin (TBG) promoter and having an AAV8 capsid protein, and rAAV vectors encoding artificial microRNAs (amiRs) targeting Slc16A2, Acsl5, Cyb5b, and combinations thereof.

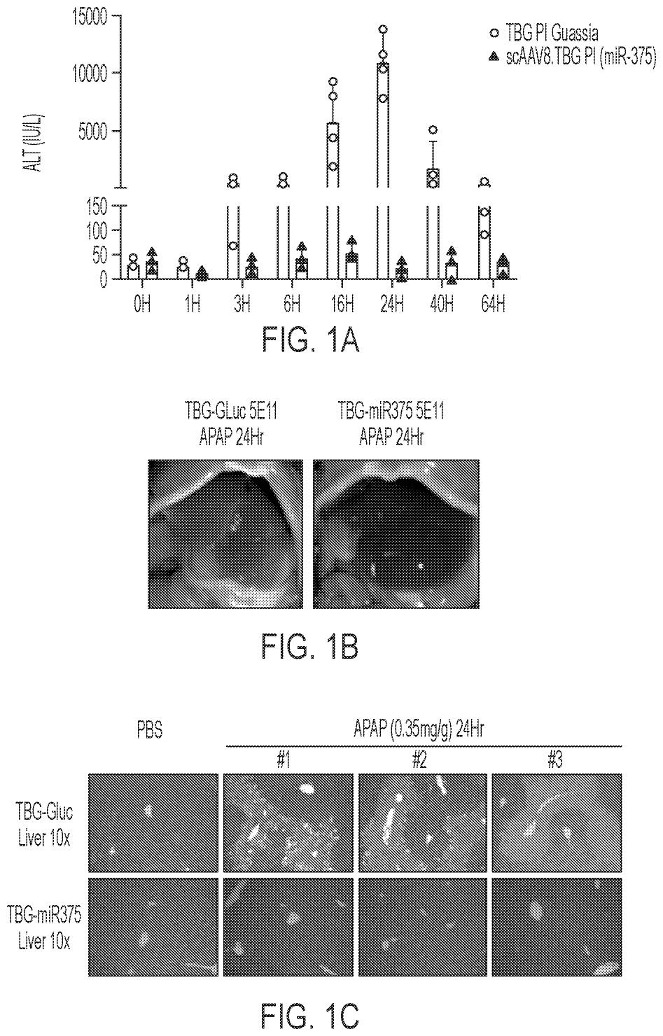

FIGS. 1A-1C show liver-specific miR-375 expression in mice inhibits acetaminophen (APAP)-induced liver damage. FIG. 1A shows serum alanine aminotransferase (ALT) levels in acetaminophen overdosed mice for different hours. (Control: TBG-PI Guassia; miR-375: scAAV8-TBG-PI-miR-375 (TBG-miR375)). FIG. 1B shows representative gross images of livers from overdosed mice after 24 hours (injected virus dose: 5E11 genome copy per mice). FIG. 1C shows representative images of hematoxylin and eosin (H&E) stained liver sections of mice 24 hours after acetaminophen administration (#1, #2, #3 indicate 3 individual animals. Magnification, 10×).

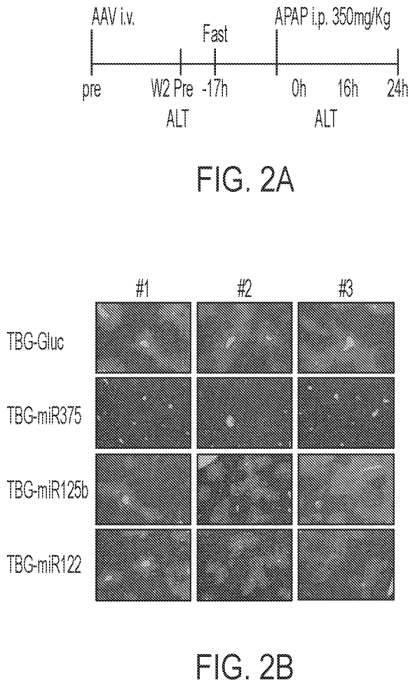

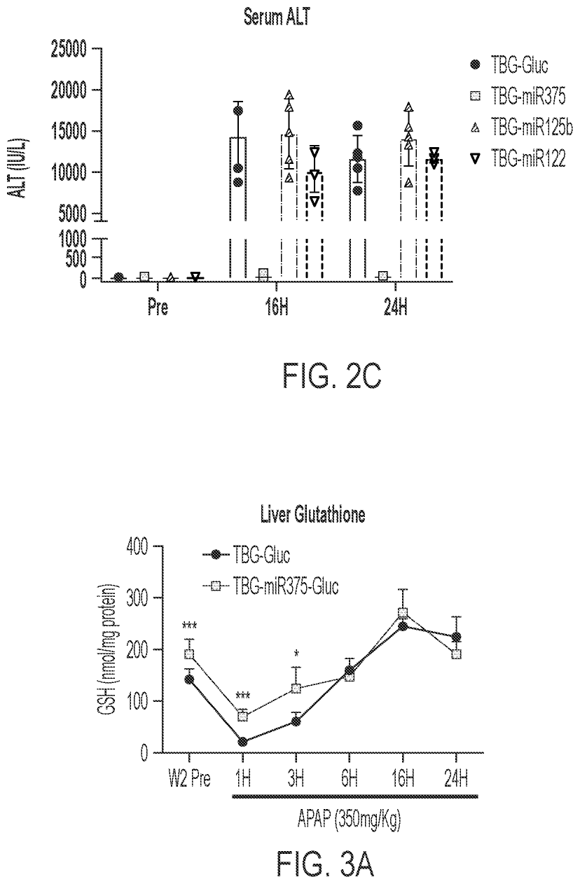

FIGS. 2A-2C show data relating to a protection efficacy comparison among AAV-mediated liver-specific delivery of miR-375, miR-125b, or miR-122 in APAP-ALF model. FIG. 2A shows a schematic representation of the experimental design in APAP-induced ALF model. AAV-miRs were injected 2 weeks before the induction of ALF, animals were fast over-night before APAP administration. FIG. 2B shows representative images of H&E stained liver sections of mice 24 hours after acetaminophen administration (#1, #2, #3 indicate 3 individual animals. Magnification, 10×). FIG. 2C shows serum ALT levels in APAP overdosed mice at different time points.

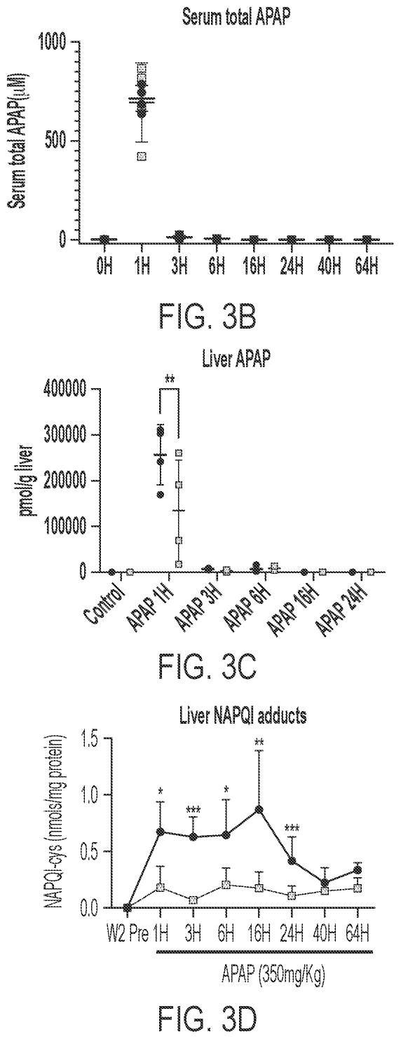

FIGS. 3A-3D show TBG-miR375 affects hepatic GSH and APAP metabolism. The levels of hepatic glutathione (GSH; FIG. 3A), serum APAP (FIG. 3B), liver APAP (FIG. 3C), and N-acetyl-p-benzoquinone imine (NAPQI)-protein adduct (FIG. 3D) in acetaminophen-overdosed mice at different time points are shown (Control: TBG-Gluc black dot; TBG-miR375 squares).

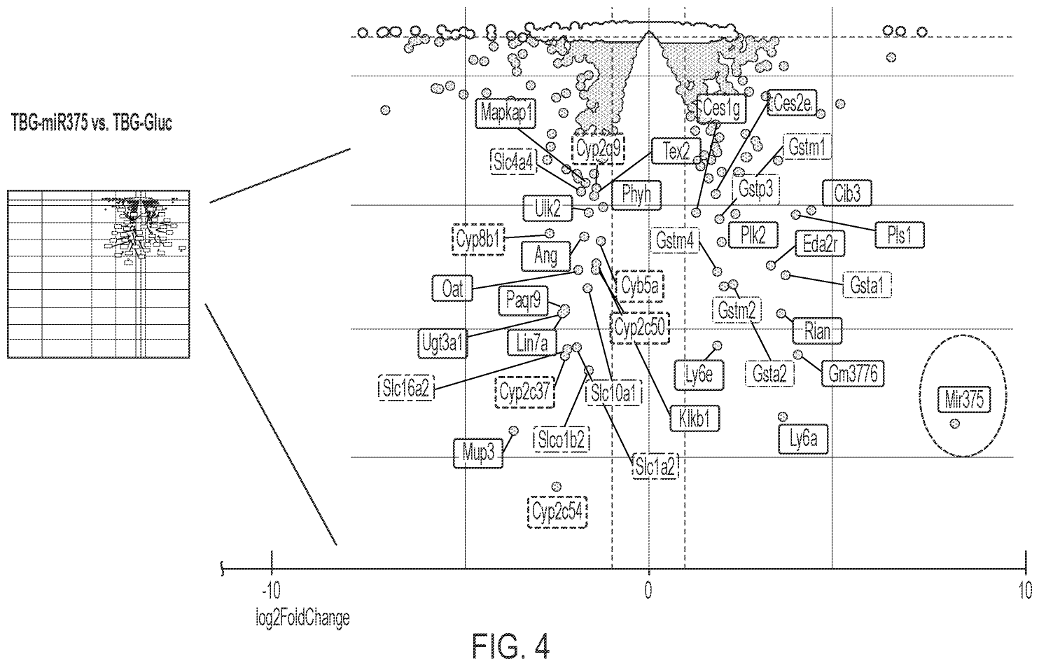

RNA-seq data from liver transduced with AAV8-miR-375 demonstrates that genes regulated by miR-375 are enriched in the cytochrome P450 system (CYP), APAP-metabolizing enzymes responsible for cytotoxic byproduct NAPQI (N-acetyl-p-benzoquinone imine) formation, as well as NAPQI-scavenging Glutathione-S-transferases (GST) and Sulfotransferases (SULT). FIG. 4 shows a volcano plot from RNA-seq data comparing liver transcriptome change between TBG-miR375 and TBG-Gluc expressing mice (Cytochome P450 (CYP) genes are highlighted on the left; Glutathione-S-transferase (GST) genes are highlighted on the right; solute carrier family (SLC) genes are darker shaded).

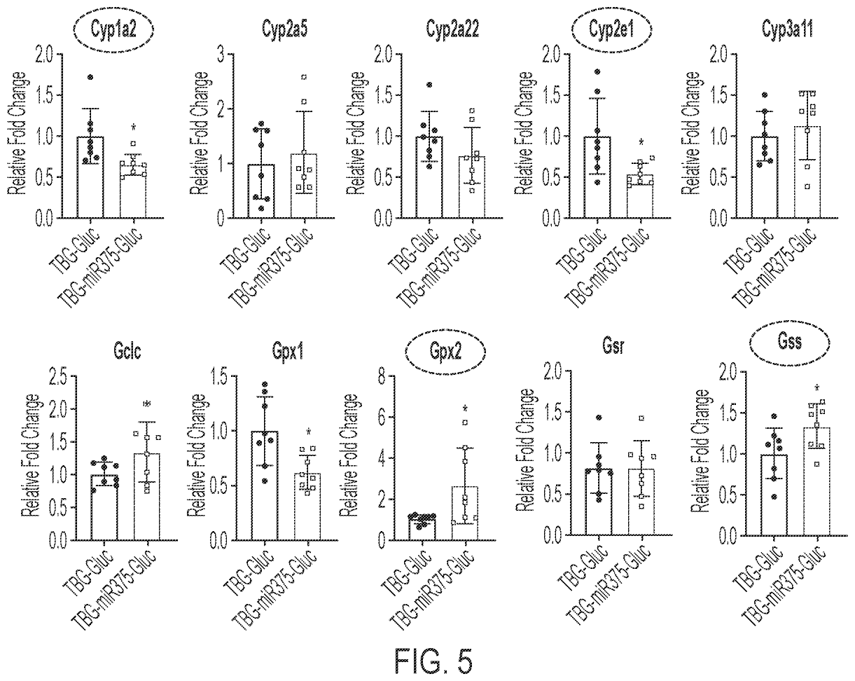

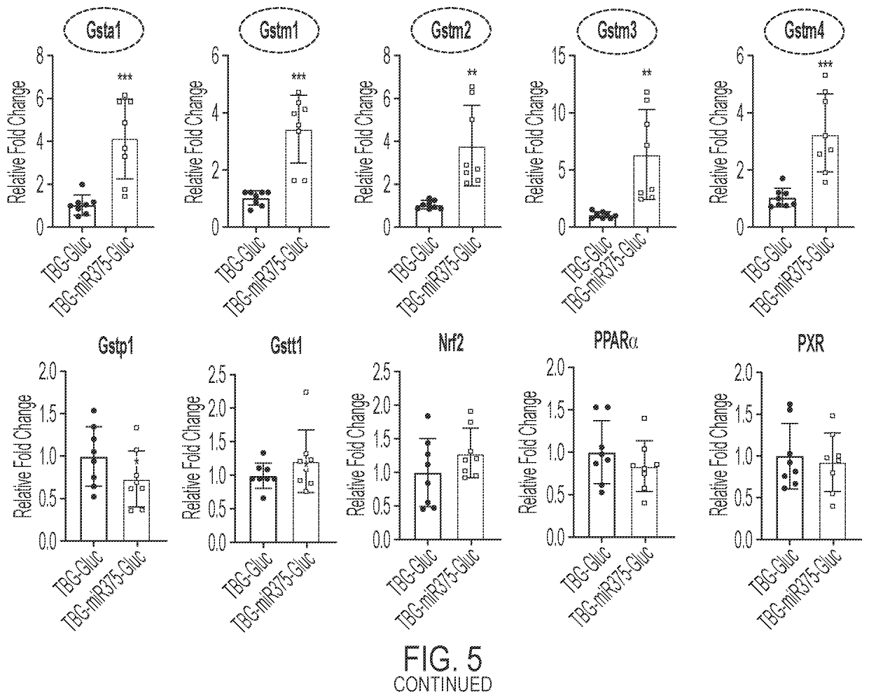

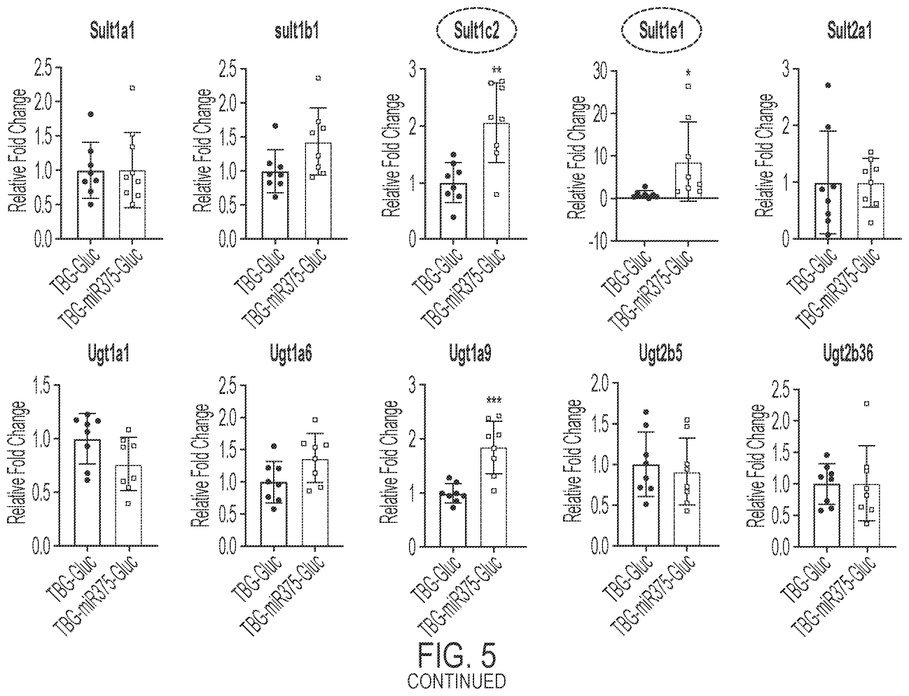

FIG. 5 shows qRT-PCR analysis of APAP metabolism genes expression in mice treated with AAV-TBG-miR-375 (right bar) or control AAV-Gluc (left bar). *P<0.05, **P<0.01, ***P<0.001 versus control (two-tailed t-test).

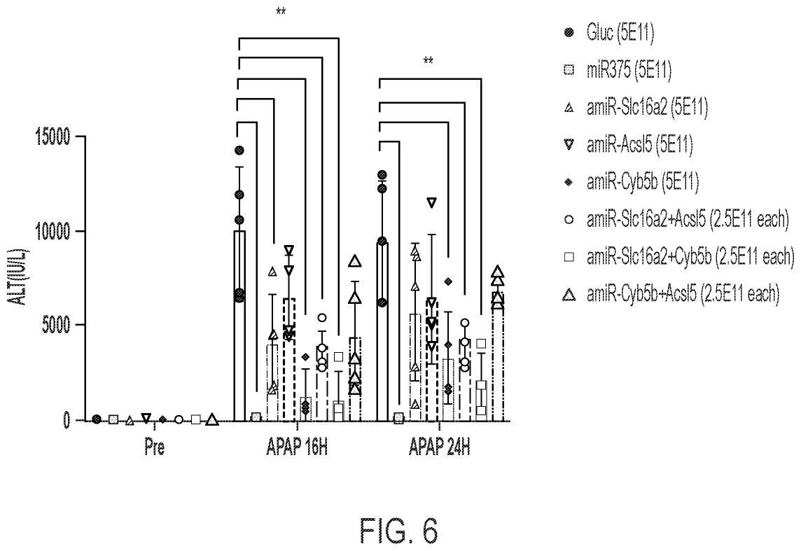

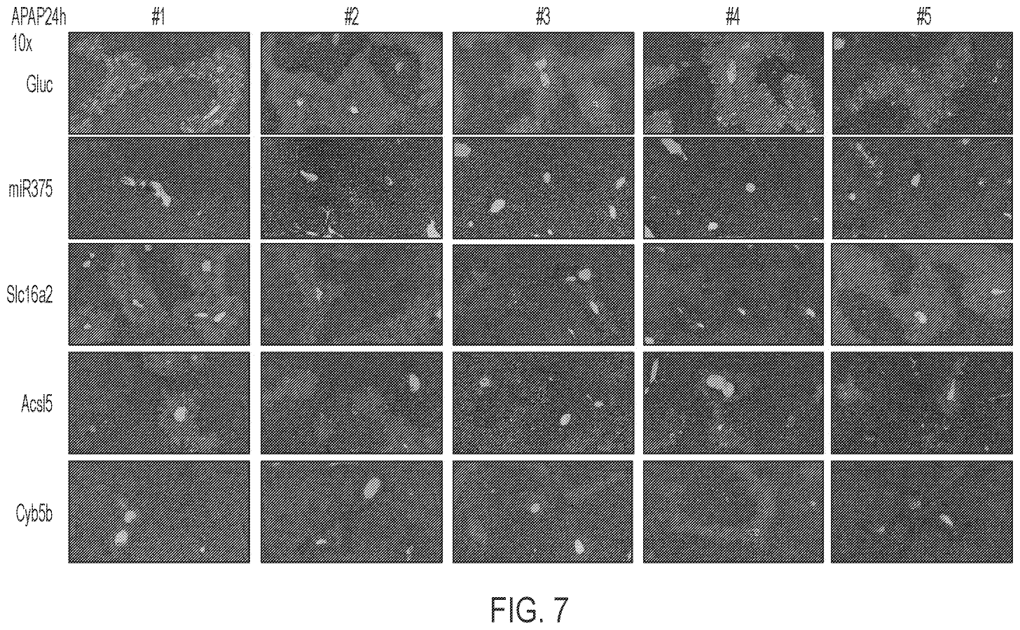

FIG. 6 shows data evaluating the contributions of each individual or combination of miR-375 target genes in APAP-ALF inhibition by AAV-mediated artificial microRNA scaffold housing small interfering RNA (siRNA) for gene silencing (amiR). Serum ALT levels in APAP overdosed mice pre-expressing AAV-TBG-miR-375 or indicating AAV-TBG-amiR vectors. Data indicate that inhibition of miR-375 target genes (e.g., Slc16a2, Cyb5b and Acsl5) by amiRNAs partially mimic miR-375-mediated protection. Complete recapitulation of the protective effects from miR-375 can be achieved in the combined administration of these three AAV-amiR vectors.

FIG. 7 shows representative images of H&E stained liver sections of mice as treated in FIG. 6 at 24 hours after APAP administration (#1-#5 indicate 5 individual animals. Magnification, 10×).

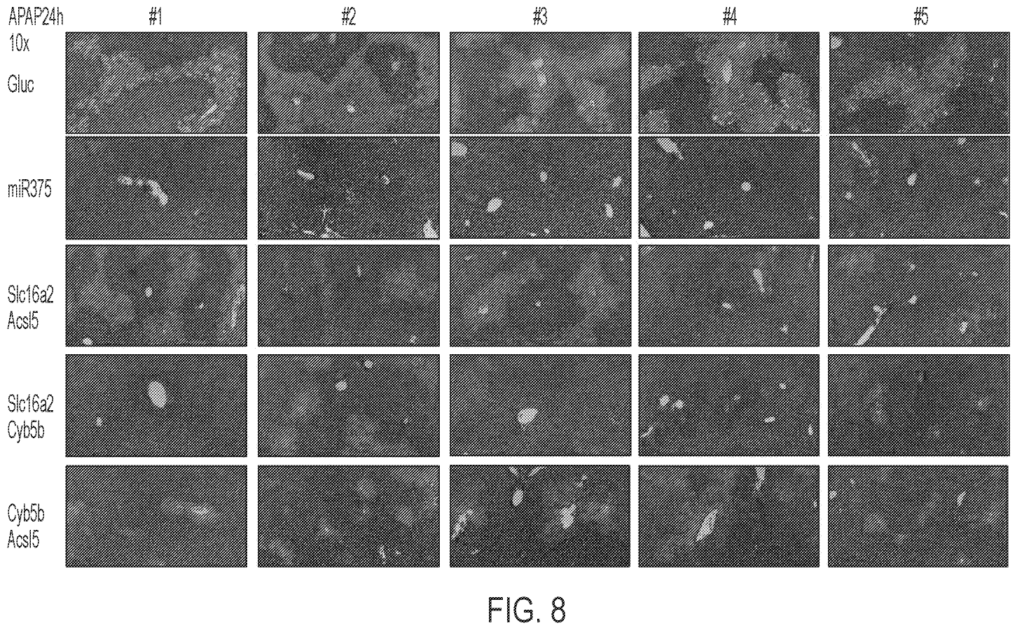

FIG. 8 shows representative images of H&E stained liver sections of mice as treated in FIG. 6 at 24 hours after APAP administration (#1-#5 indicate 5 individual animals. Magnification, 10×).

### EQUIVALENTS

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

### SEQUENCES

In some embodiments, an isolated nucleic acid or vector (e.g., rAAV vector) described by the disclosure comprises or consists of a sequence set forth in any one of SEQ ID NOs: 1-12. In some embodiments, an isolated nucleic acid or vector (e.g., rAAV vector) described by the disclosure comprises or consists of a sequence set forth in any one of SEQ ID NOs: 9-12, wherein the sequence corresponding to a reporter protein (e.g., Guassia, Gluc, etc.) has been removed. In some embodiments, an isolated nucleic acid or vector (e.g., rAAV vector) described by the disclosure comprises or consists of a sequence that is complementary (e.g., the complement of) a sequence set forth in any one of SEQ ID NOs: 1-12. In some embodiments, an isolated nucleic acid or vector (e.g., rAAV vector) described by the disclosure comprises or consists of a sequence that is a reverse complement of a sequence set forth in any one of SEQ ID NOs: 1-12. In some embodiments, an isolated nucleic acid or vector (e.g., rAAV vector) described by the disclosure comprises or consists of a portion of a sequence set forth in any one of SEQ ID NOs: 1-12. A portion may comprise at least 25%, 50%, 60%, 70%, 80%, 90%, 95%, or 99% of a sequence set forth in any one of SEQ ID NOs: 1-12. In some embodiments, a nucleic acid sequence described by the disclosure is a nucleic acid sense strand (e.g., 5′ to 3′ strand), or in the context of a viral sequences a plus (+) strand. In some embodiments, a nucleic acid sequence described by the disclosure is a nucleic acid antisense strand (e.g., 3′ to 5′ strand), or in the context of viral sequences a minus (−) strand.

The skilled artisan recognizes that when referring to nucleic acid sequences comprising or encoding inhibitory nucleic acids (e.g., dsRNA, siRNA, miRNA, amiRNA, etc.) any one or more thymidine (T) nucleotides or uridine (U) nucleotides in a sequence provided herein may be replaced with any other nucleotide suitable for base pairing (e.g., via a Watson-Crick base pair) with an adenosine nucleotide. For example, T may be replaced with U, and U may be replaced with T.

All NCBI Gene and Accession Number Sequences are incorporated herein by reference in their entireties.

The application contains a Sequence Listing submitted, which is incorporated herein by reference. The Sequence Listing is provided as a Text File entitled U0120.70136WO00-SEQ.txt, created on Apr. 9, 2021, and is 15,534 bytes in size.

>miR-375 antisense
(SEQ ID NO: 1)
UUUGUUCGUUCGGCUCGCGUGA
>pri-miR-375
(SEQ ID NO: 2)
ACCGCGGTGCTCAGGTGAGAGCGGCGGCTAGCGGGAGCGCTGTGCACTCGAGGAA
GCTCATCCACCAGACACCGCCGACGACTGCCGCCCGGCCCCGGGTCTTCCGCTCCG
GCCCCGCGACGAGCCCCTCGCACAAACCGGACCTGAGCGTTTTGTTCGTTCGGCTCG
CGTGAGGCAGGGGCGGCTTCTCAGCATCAGCCTTGGGGCCGGCCAGATCGCCATGC
AAACACCAGCCGCCGTCGCCACCGCCACCATATGGGTCGCAGAGACTGAGCACGGT
CCC
>Slc16a2 antisense
(SEQ ID NO: 3)
UGAUAUUCCUGGUUUGCCUCC
>amiR Slc16a2
(SEQ ID NO: 4)
AGGGCTCTGCGTTTGCTCCAGGTAGTCCGCTGCTCCCTTGGGCCTGGGCCCACTGAC
AGCCCTGGTGCCTCTGGCCGGCTGCACACCTCCTGGCGGGCAGCTGTGTGATATTCC
TGGTTTGCCTCCTGTTCTGGCAATACCTGGGAGGCAATGCGGGAATATCACACGGA
GGCCTGCCCTGACTGCCCACGGTGCCGTGGCCAAAGAGGATCTAAGGGCACCGCTG
AGGGCCTACCTAACCATCGTGGGGAATAAGGACAGTGTCACCCG
>Acsl5 antisense
(SEQ ID NO: 5)
UUCAUACAACGUCUUGGCGUC
>amiR Acsl5
(SEQ ID NO: 6)
AGGGCTCTGCGTTTGCTCCAGGTAGTCCGCTGCTCCCTTGGGCCTGGGCCCACTGAC
AGCCCTGGTGCCTCTGGCCGGCTGCACACCTCCTGGCGGGCAGCTGTGTTCATACAA
CGTCTTGGCGTCTGTTCTGGCAATACCTGGACGCCAACTCATTGTATGAACACGGAG
GCCTGCCCTGACTGCCCACGGTGCCGTGGCCAAAGAGGATCTAAGGGCACCGCTGA
GGGCCTACCTAACCATCGTGGGGAATAAGGACAGTGTCACCCG
>Cyb5b antisense
(SEQ ID NO: 7)
UUUAAGGUCACUCGGAUGGAC
>amiR Cyb5b
(SEQ ID NO: 8)
AGGGCTCTGCGTTTGCTCCAGGTAGTCCGCTGCTCCCTTGGGCCTGGGCCCACTGAC
AGCCCTGGTGCCTCTGGCCGGCTGCACACCTCCTGGCGGGCAGCTGTGTTTAAGGTC
ACTCGGATGGACTGTTCTGGCAATACCTGGTCCATCCCTGCGACCTTAAACACGGAG
GCCTGCCCTGACTGCCCACGGTGCCGTGGCCAAAGAGGATCTAAGGGCACCGCTGA
GGGCCTACCTAACCATCGTGGGGAATAAGGACAGTGTCACCCG
>pAAVsc-TBG-PI(MiR-375)-Guassia
(SEQ ID NO: 9)
ctgcgcgctcgctcgctcactgaggccgcccgggcaaagcccgggcgtcgggcgacctttggtcgcccggcctcagtgagcgagcga
gcgcgcagagagggagtgtagccatgctctaggaagatcaattcggtacaattcacgcgtactagtaggttaatttttaaaaagcagtcaaa
agtccaagtggcccttggcagcatttactctctctgtttgctctggttaataatctcaggagcacaaacattccgctagcagatccaggttaattt
ttaaaaagcagtcaaaagtccaagtggcccttggcagcatttactctctctgtttgctctggttaataatctcaggagcacaaacattccggtac
cccatgggggctggaagctacctttgacatcatttcctctgcgaatgcatgtataatttctacagaacctattagaaaggatcacccagcctct
gcttttgtacaactttcccttaaaaaactgccaattccactgctgtttggcccaatagtgagaactttttcctgctgcctcttggtgcttttgcctatg
gcccctattctgcctgctgaagacactcttgccagcatggacttaaacccctccagctctgacaatcctctttctcttttgttttacatgaagggtc
tggcagccaaagcaatcactcaaagttcaaaccttatcattttttgctttgttcctcttggccttggttttgtacatcagctttgaaaataccatccc
agggttaatgctggggttaatttataactaagagtgctctagttttgcaatacaggacatgctataaaaatggaaagatatttgtggcggcccta
gagtcgatcgaggaactgaaaaaccagaaagttaactggtaagtttagtctttttgtcttttatttcagaccgcggtgctcaggtgagagcggc
ggctagcgggagcgctgtgcactcgaggaagctcatccaccagacaccgccgacgactgccgcccggccccgggtcttccgctccggc
cccgcgacgagcccctcgcacaaaccggacctgagcgttttgttcgttcggctcgcgtgaggcaggggcggcttctcagcatcagccttg
gggccggccagatcgccatgcaaacaccagccgccgtcgccaccgccaccatatgggtcgcagagactgagcacggtcccgtcccgg
atccggtggtggtgcaaatcaaagaactgctcctcagtggatgttgcctttacttctaggcctgtacggaagtgttacttctgctctaaaagctg
cggaattgtacccgcggccgatccaccggtcgccaccatctagcatgggagtcaaagttctgtttgccctgatctgcatcgctgtggccgag
gccaagcccaccgagaacaacgaagacttcaacatcgtggccgtggccagcaacttcgcgaccacggatctcgatgctgaccgcggga
agttgcccggcaagaagctgccgctggaggtgctcaaagagatggaagccaatgcccggaaagctggctgcaccaggggctgtctgat
ctgcctgtcccacatcaagtgcacgcccaagatgaagaagttcatcccaggacgctgccacacctacgaaggcgacaaagagtccgcac
agggcggcataggcgaggcgatcgtcgacattcctgagattcctgggttcaaggacttggagcccatggagcagttcatcgcacaggtcg
atctgtgtgtggactgcacaactggctgcctcaaagggcttgccaacgtgcagtgttctgacctgctcaagaagtggctgccgcaacgctgt
gcgacctttgccagcaagatccagggccaggtggacaagatcaagggggccggtggtgactagctcgacgctgccgccgatatcagag
ctcctgcaggtcgactctagagatctttttccctctgccaaaaattatggggacatcatgaagccccttgagcatctgacttctggctaataaag
gaaatttattttcattgcaatagtgtgttggaattttttgtgtctctcactcgcatatgaacctaggtagataagtagcatggcgggttaatcattaa
ctacaaggaacccctagtgatggagttggccactccctctctgcgcgctcgctcgctcactgaggccgggcgaccaaaggtcgcccgac
gcccgggctttgcccgggcggcctcagtgagcgagcgagcgcgcag
>pAAVsc-TBG-(amiR-Slc16a2-1)-Gluc-bGH
(SEQ ID NO: 10)
ctgcgcgctcgctcgctcactgaggccgcccgggcaaagcccgggcgtcgggcgacctttggtcgcccggcctcagtgagcgagcga
gcgcgcagagagggagtgtagccatgctctaggaagatcaattcggtacaattcacgcgtactagtaggttaatttttaaaaagcagtcaaa
agtccaagtggcccttggcagcatttactctctctgtttgctctggttaataatctcaggagcacaaacattccgctagcagatccaggttaattt
ttaaaaagcagtcaaaagtccaagtggcccttggcagcatttactctctctgtttgctctggttaataatctcaggagcacaaacattccggtac
cccatgggggctggaagctacctttgacatcatttcctctgcgaatgcatgtataatttctacagaacctattagaaaggatcacccagcctct
gcttttgtacaactttcccttaaaaaactgccaattccactgctgtttggcccaatagtgagaactttttcctgctgcctcttggtgcttttgcctatg
gcccctattctgcctgctgaagacactcttgccagcatggacttaaacccctccagctctgacaatcctctttctcttttgttttacatgaagggtc
tggcagccaaagcaatcactcaaagttcaaaccttatcattttttgctttgttcctcttggccttggttttgtacatcagctttgaaaataccatccc
agggttaatgctggggttaatttataactaagagtgctctagttttgcaatacaggacatgctataaaaatggaaagatatttgtggcggcccta
gagtcgatcgaggaactgaaaaaccagaaagttaactggtaagtttagtctttttgtcttttatttcaggtcccagatcAGGGCTCTGC
GTTTGCTCCAGGTAGTCCGCTGCTCCCTTGGGCCTGGGCCCACTGACAGCCCTGGTG
CCTCTGGCCGGCTGCACACCTCCTGGCGGGCAGCTGTGTGATATTCCTGGTTTGCCT
CCTGTTCTGGCAATACCTGGGAGGCAATGCGGGAATATCACACGGAGGCCTGCCCT
GACTGCCCACGGTGCCGTGGCCAAAGAGGATCTAAGGGCACCGCTGAGGGCCTACC
TAACCATCGTGGGGAATAAGGACAGTGTCACCCGggggatccggtggtggtgcaaatcaaagaactgct
cctcagtggatgttgcctttacttctaggcctgtacggaagtgttacttctgctctaaaagctgcggaattgtacccgcggccgatccaccggt
cgccaccatctagcatgggagtcaaagttctgtttgccctgatctgcatcgctgtggccgaggccaagcccaccgagaacaacgaagactt
caacatcgtggccgtggccagcaacttcgcgaccacggatctcgatgctgaccgcgggaagttgcccggcaagaagctgccgctggag
gtgctcaaagagatggaagccaatgcccggaaagctggctgcaccaggggctgtctgatctgcctgtcccacatcaagtgcacgcccaa
gatgaagaagttcatcccaggacgctgccacacctacgaaggcgacaaagagtccgcacagggcggcataggcgaggcgatcgtcga
cattcctgagattcctgggttcaaggacttggagcccatggagcagttcatcgcacaggtcgatctgtgtgtggactgcacaactggctgcct
caaagggcttgccaacgtgcagtgttctgacctgctcaagaagtggctgccgcaacgctgtgcgacctttgccagcaagatccagggcca
ggtggacaagatcaagggggccggtggtgactagctcgacgctgatcagcctcgactgtgccttctagttgccagccatctgttgtttgccc
ctcccccgtgccttccttgaccctggaaggtgccactcccactgtcctttcctaataaaatgaggaaattgcatcgcattgtctgagtaggtgtc
attctattctggggggtggggtggggcaggacagcaagggggaggattgggaagacaattaggtagataagtagcatggcgggttaatc
attaactacaaggaacccctagtgatggagttggccactccctctctgcgcgctcgctcgctcactgaggccgggcgaccaaaggtcgcc
cgacgcccgggctttgcccgggcggcctcagtgagcgagcgagcgcgcag
>pAAVsc-TBG(amiR-Acsl5-1)-Gluc-bGH
(SEQ ID NO: 11)
ctgcgcgctcgctcgctcactgaggccgcccgggcaaagcccgggcgtcgggcgacctttggtcgcccggcctcagtgagcgagcga
gcgcgcagagagggagtgtagccatgctctaggaagatcaattcggtacaattcacgcgtactagtaggttaatttttaaaaagcagtcaaa
agtccaagtggcccttggcagcatttactctctctgtttgctctggttaataatctcaggagcacaaacattccgctagcagatccaggttaattt
ttaaaaagcagtcaaaagtccaagtggcccttggcagcatttactctctctgtttgctctggttaataatctcaggagcacaaacattccggtac
cccatgggggctggaagctacctttgacatcatttcctctgcgaatgcatgtataatttctacagaacctattagaaaggatcacccagcctct
gcttttgtacaactttcccttaaaaaactgccaattccactgctgtttggcccaatagtgagaactttttcctgctgcctcttggtgcttttgcctatg
gcccctattctgcctgctgaagacactcttgccagcatggacttaaacccctccagctctgacaatcctctttctcttttgttttacatgaagggtc
tggcagccaaagcaatcactcaaagttcaaaccttatcattttttgctttgttcctcttggccttggttttgtacatcagctttgaaaataccatccc
agggttaatgctggggttaatttataactaagagtgctctagttttgcaatacaggacatgctataaaaatggaaagatatttgtggcggcccta
gagtcgatcgaggaactgaaaaaccagaaagttaactggtaagtttagtctttttgtcttttatttcaggtcccagatcAGGGCTCTGC
GTTTGCTCCAGGTAGTCCGCTGCTCCCTTGGGCCTGGGCCCACTGACAGCCCTGGTG
CCTCTGGCCGGCTGCACACCTCCTGGCGGGCAGCTGTGTTCATACAACGTCTTGGCG
TCTGTTCTGGCAATACCTGGACGCCAACTCATTGTATGAACACGGAGGCCTGCCCTG
ACTGCCCACGGTGCCGTGGCCAAAGAGGATCTAAGGGCACCGCTGAGGGCCTACCT
AACCATCGTGGGGAATAAGGACAGTGTCACCCGggggatccggtggtggtgcaaatcaaagaactgctc
ctcagtggatgttgcctttacttctaggcctgtacggaagtgttacttctgctctaaaagctgcggaattgtacccgcggccgatccaccggtc
gccaccatctagcatgggagtcaaagttctgtttgccctgatctgcatcgctgtggccgaggccaagcccaccgagaacaacgaagacttc
aacatcgtggccgtggccagcaacttcgcgaccacggatctcgatgctgaccgcgggaagttgcccggcaagaagctgccgctggagg
tgctcaaagagatggaagccaatgcccggaaagctggctgcaccaggggctgtctgatctgcctgtcccacatcaagtgcacgcccaag
atgaagaagttcatcccaggacgctgccacacctacgaaggcgacaaagagtccgcacagggcggcataggcgaggcgatcgtcgac
attcctgagattcctgggttcaaggacttggagcccatggagcagttcatcgcacaggtcgatctgtgtgtggactgcacaactggctgcctc
aaagggcttgccaacgtgcagtgttctgacctgctcaagaagtggctgccgcaacgctgtgcgacctttgccagcaagatccagggccag
gtggacaagatcaagggggccggtggtgactagctcgacgctgatcagcctcgactgtgccttctagttgccagccatctgttgtttgcccc
tcccccgtgccttccttgaccctggaaggtgccactcccactgtcctttcctaataaaatgaggaaattgcatcgcattgtctgagtaggtgtca
ttctattctggggggtggggtggggcaggacagcaagggggaggattgggaagacaattaggtagataagtagcatggcgggttaatcat
taactacaaggaacccctagtgatggagttggccactccctctctgcgcgctcgctcgctcactgaggccgggcgaccaaaggtcgcccg
acgcccgggctttgcccgggcggcctcagtgagcgagcgagcgcgcag
>pAAVsc-TBG(amiR-Cyb5b-2)-Gluc-bGH
(SEQ ID NO: 12)
ctgcgcgctcgctcgctcactgaggccgcccgggcaaagcccgggcgtcgggcgacctttggtcgcccggcctcagtgagcgagcga
gcgcgcagagagggagtgtagccatgctctaggaagatcaattcggtacaattcacgcgtactagtaggttaatttttaaaaagcagtcaaa
agtccaagtggcccttggcagcatttactctctctgtttgctctggttaataatctcaggagcacaaacattccgctagcagatccaggttaattt
ttaaaaagcagtcaaaagtccaagtggcccttggcagcatttactctctctgtttgctctggttaataatctcaggagcacaaacattccggtac
cccatgggggctggaagctacctttgacatcatttcctctgcgaatgcatgtataatttctacagaacctattagaaaggatcacccagcctct
gcttttgtacaactttcccttaaaaaactgccaattccactgctgtttggcccaatagtgagaactttttcctgctgcctcttggtgcttttgcctatg
gcccctattctgcctgctgaagacactcttgccagcatggacttaaacccctccagctctgacaatcctctttctcttttgttttacatgaagggtc
tggcagccaaagcaatcactcaaagttcaaaccttatcattttttgctttgttcctcttggccttggttttgtacatcagctttgaaaataccatccc
agggttaatgctggggttaatttataactaagagtgctctagttttgcaatacaggacatgctataaaaatggaaagatatttgtggcggcccta
gagtcgatcgaggaactgaaaaaccagaaagttaactggtaagtttagtctttttgtcttttatttcaggtcccagatcAGGGCTCTGC
GTTTGCTCCAGGTAGTCCGCTGCTCCCTTGGGCCTGGGCCCACTGACAGCCCTGGTG
CCTCTGGCCGGCTGCACACCTCCTGGCGGGCAGCTGTGTTTAAGGTCACTCGGATGG
ACTGTTCTGGCAATACCTGGTCCATCCCTGCGACCTTAAACACGGAGGCCTGCCCTG
ACTGCCCACGGTGCCGTGGCCAAAGAGGATCTAAGGGCACCGCTGAGGGCCTACCT
AACCATCGTGGGGAATAAGGACAGTGTCACCCGggggatccggtggtggtgcaaatcaaagaactgctc
ctcagtggatgttgcctttacttctaggcctgtacggaagtgttacttctgctctaaaagctgcggaattgtacccgcggccgatccaccggtc
gccaccatctagcatgggagtcaaagttctgtttgccctgatctgcatcgctgtggccgaggccaagcccaccgagaacaacgaagacttc
aacatcgtggccgtggccagcaacttcgcgaccacggatctcgatgctgaccgcgggaagttgcccggcaagaagctgccgctggagg
tgctcaaagagatggaagccaatgcccggaaagctggctgcaccaggggctgtctgatctgcctgtcccacatcaagtgcacgcccaag
atgaagaagttcatcccaggacgctgccacacctacgaaggcgacaaagagtccgcacagggcggcataggcgaggcgatcgtcgac
attcctgagattcctgggttcaaggacttggagcccatggagcagttcatcgcacaggtcgatctgtgtgtggactgcacaactggctgcctc
aaagggcttgccaacgtgcagtgttctgacctgctcaagaagtggctgccgcaacgctgtgcgacctttgccagcaagatccagggccag
gtggacaagatcaagggggccggtggtgactagctcgacgctgatcagcctcgactgtgccttctagttgccagccatctgttgtttgcccc
tcccccgtgccttccttgaccctggaaggtgccactcccactgtcctttcctaataaaatgaggaaattgcatcgcattgtctgagtaggtgtca
ttctattctggggggtggggtggggcaggacagcaagggggaggattgggaagacaattaggtagataagtagcatggcgggttaatcat
taactacaaggaacccctagtgatggagttggccactccctctctgcgcgctcgctcgctcactgaggccgggcgaccaaaggtcgcccg
acgcccgggctttgcccgggcggcctcagtgagcgagcgagcgcgcag

1. A method for inhibiting cytochrome p450-mediated toxicity in a cell, the method comprising administering to the cell an isolated nucleic acid encoding one or more miR-375 molecules.

2. The method of claim 1, wherein the cytochrome p450-mediated toxicity is a result of acetaminophen (APAP) overdose.

3. The method of claim 1, wherein the cell is in a subject, optionally wherein the subject is a human.

4. The method of claim 3, wherein the subject has acute liver failure (ALF).

5. (canceled)

6. The method of claim 1, wherein the miR-375 molecule comprises the sequence set forth in SEQ ID NO: 1 or is encoded by the sequence set forth in SEQ ID NO: 2.

7. The method of claim 1, wherein the isolated nucleic acid encoding the miR-375 molecule is an artificial miRNA (amiRNA).

8. The method of claim 1, wherein the isolated nucleic acid is flanked by adeno-associated virus (AAV) inverted terminal repeats (ITRs).

9-10. (canceled)

11. The method of claim 1, wherein the isolated nucleic acid encoding the one or more miR-375 molecules is encapsidated by an AAV capsid protein, optionally wherein the AAV capsid protein is an AAV8 capsid protein.

12-18. (canceled)

19. An artificial microRNA (amiRNA) comprising a miRNA backbone flanking an isolated nucleic acid encoding a miR-375 molecule.

20. The amiRNA of claim 19, wherein the miR-375 molecule comprises the sequence set forth in SEQ ID NO: 1.

21-26. (canceled)

27. A recombinant adeno-associated virus (rAAV) comprising:
(i) the amiRNA of claim 19; and
(ii) an AAV capsid protein.

28. The rAAV of claim 27, wherein the capsid protein is an AAV8 capsid protein.

29-49. (canceled)

50. An isolated nucleic acid comprising an expression cassette encoding two or more artificial miRNAs (amiRNAs), wherein each of the two or more amiRNAs is independently selected from: a miR-375 amiRNA, an amiRNA targeting Slc16a2, an amiRNA targeting Acsl5, and an amiRNA targeting Cyb5b.

51. The isolated nucleic acid of claim 50, wherein the miR-375 amiRNA comprises the sequence set forth in SEQ ID NO: 1 or is encoded by the sequence set forth in SEQ ID NO: 2.

52. The isolated nucleic acid of claim 50, wherein the amiRNA targeting Slc16a2 comprises the sequence set forth in SEQ ID NO: 3 or is encoded by the sequence set forth in SEQ ID NO: 4.

53. The isolated nucleic acid of claim 50, wherein the amiRNA targeting Acsl5 comprises the sequence set forth in SEQ ID NO: 5 or is encoded by the sequence set forth in SEQ ID NO: 6.

54. The isolated nucleic acid of claim 50, wherein the amiRNA targeting Cyb5b comprises the sequence set forth in SEQ ID NO: 7 or is encoded by the sequence set forth in SEQ ID NO: 8.

55-56. (canceled)

57. The isolated nucleic acid of claim 50, wherein the expression cassette is flanked by adeno-associated virus (AAV) inverted terminal repeats (ITRs).

58-59. (canceled)

60. A recombinant adeno-associated virus (rAAV) comprising:
(i) the rAAV vector of claim 50; and
(ii) an AAV capsid protein.

61. (canceled)

62. A method for inhibiting cytochrome p450-mediated toxicity in a subject, the method comprising administering to the subject the or rAAV of claim 60.

63-66. (canceled)


SEQUENCE LISTING (12 sequences)

SEQ ID NO: 1 - RNA, 22 nt, Artificial Sequence
  Synthetic
uuuguucguucggcucgcguga

SEQ ID NO: 2 - DNA, 283 bp, Artificial Sequence
  Synthetic
accgcggtgctcaggtgagagcggcggctagcgggagcgctgtgcactcgaggaagctcatccaccagacaccgccgacgactgccgcccggccccgggtcttccgctccggccccgcgacgagcccctcgcacaaaccggacctgagcgttttgttcgttcggctcgcgtgaggcaggggcggcttctcagcatcagccttggggccggccagatcgccatgcaaacaccagccgccgtcgccaccgccaccatatgggtcgcagagactgagcacggtccc

SEQ ID NO: 3 - RNA, 21 nt, Artificial Sequence
  Synthetic
ugauauuccugguuugccucc

SEQ ID NO: 4 - DNA, 270 bp, Artificial Sequence
  Synthetic
agggctctgcgtttgctccaggtagtccgctgctcccttgggcctgggcccactgacagccctggtgcctctggccggctgcacacctcctggcgggcagctgtgtgatattcctggtttgcctcctgttctggcaatacctgggaggcaatgcgggaatatcacacggaggcctgccctgactgcccacggtgccgtggccaaagaggatctaagggcaccgctgagggcctacctaaccatcgtggggaataaggacagtgtcacccg

SEQ ID NO: 5 - RNA, 21 nt, Artificial Sequence
  Synthetic
uucauacaacgucuuggcguc

SEQ ID NO: 6 - DNA, 270 bp, Artificial Sequence
  Synthetic
agggctctgcgtttgctccaggtagtccgctgctcccttgggcctgggcccactgacagccctggtgcctctggccggctgcacacctcctggcgggcagctgtgttcatacaacgtcttggcgtctgttctggcaatacctggacgccaactcattgtatgaacacggaggcctgccctgactgcccacggtgccgtggccaaagaggatctaagggcaccgctgagggcctacctaaccatcgtggggaataaggacagtgtcacccg

SEQ ID NO: 7 - RNA, 21 nt, Artificial Sequence
  Synthetic
uuuaaggucacucggauggac

SEQ ID NO: 8 - DNA, 270 bp, Artificial Sequence
  Synthetic
agggctctgcgtttgctccaggtagtccgctgctcccttgggcctgggcccactgacagccctggtgcctctggccggctgcacacctcctggcgggcagctgtgtttaaggtcactcggatggactgttctggcaatacctggtccatccctgcgaccttaaacacggaggcctgccctgactgcccacggtgccgtggccaaagaggatctaagggcaccgctgagggcctacctaaccatcgtggggaataaggacagtgtcacccg

SEQ ID NO: 9 - DNA, 2262 bp, Artificial Sequence
  Synthetic
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcggtacaattcacgcgtactagtaggttaatttttaaaaagcagtcaaaagtccaagtggcccttggcagcatttactctctctgtttgctctggttaataatctcaggagcacaaacattccgctagcagatccaggttaatttttaaaaagcagtcaaaagtccaagtggcccttggcagcatttactctctctgtttgctctggttaataatctcaggagcacaaacattccggtaccccatgggggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatatttgtggcggccctagagtcgatcgaggaactgaaaaaccagaaagttaactggtaagtttagtctttttgtcttttatttcagaccgcggtgctcaggtgagagcggcggctagcgggagcgctgtgcactcgaggaagctcatccaccagacaccgccgacgactgccgcccggccccgggtcttccgctccggccccgcgacgagcccctcgcacaaaccggacctgagcgttttgttcgttcggctcgcgtgaggcaggggcggcttctcagcatcagccttggggccggccagatcgccatgcaaacaccagccgccgtcgccaccgccaccatatgggtcgcagagactgagcacggtcccgtcccggatccggtggtggtgcaaatcaaagaactgctcctcagtggatgttgcctttacttctaggcctgtacggaagtgttacttctgctctaaaagctgcggaattgtacccgcggccgatccaccggtcgccaccatctagcatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactagctcgacgctgccgccgatatcagagctcctgcaggtcgactctagagatctttttccctctgccaaaaattatggggacatcatgaagccccttgagcatctgacttctggctaataaaggaaatttattttcattgcaatagtgtgttggaattttttgtgtctctcactcgcatatgaacctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 10 - DNA, 2285 bp, Artificial Sequence
  Synthetic
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcggtacaattcacgcgtactagtaggttaatttttaaaaagcagtcaaaagtccaagtggcccttggcagcatttactctctctgtttgctctggttaataatctcaggagcacaaacattccgctagcagatccaggttaatttttaaaaagcagtcaaaagtccaagtggcccttggcagcatttactctctctgtttgctctggttaataatctcaggagcacaaacattccggtaccccatgggggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatatttgtggcggccctagagtcgatcgaggaactgaaaaaccagaaagttaactggtaagtttagtctttttgtcttttatttcaggtcccagatcagggctctgcgtttgctccaggtagtccgctgctcccttgggcctgggcccactgacagccctggtgcctctggccggctgcacacctcctggcgggcagctgtgtgatattcctggtttgcctcctgttctggcaatacctgggaggcaatgcgggaatatcacacggaggcctgccctgactgcccacggtgccgtggccaaagaggatctaagggcaccgctgagggcctacctaaccatcgtggggaataaggacagtgtcacccgggggatccggtggtggtgcaaatcaaagaactgctcctcagtggatgttgcctttacttctaggcctgtacggaagtgttacttctgctctaaaagctgcggaattgtacccgcggccgatccaccggtcgccaccatctagcatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactagctcgacgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 11 - DNA, 2285 bp, Artificial Sequence
  Synthetic
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcggtacaattcacgcgtactagtaggttaatttttaaaaagcagtcaaaagtccaagtggcccttggcagcatttactctctctgtttgctctggttaataatctcaggagcacaaacattccgctagcagatccaggttaatttttaaaaagcagtcaaaagtccaagtggcccttggcagcatttactctctctgtttgctctggttaataatctcaggagcacaaacattccggtaccccatgggggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatatttgtggcggccctagagtcgatcgaggaactgaaaaaccagaaagttaactggtaagtttagtctttttgtcttttatttcaggtcccagatcagggctctgcgtttgctccaggtagtccgctgctcccttgggcctgggcccactgacagccctggtgcctctggccggctgcacacctcctggcgggcagctgtgttcatacaacgtcttggcgtctgttctggcaatacctggacgccaactcattgtatgaacacggaggcctgccctgactgcccacggtgccgtggccaaagaggatctaagggcaccgctgagggcctacctaaccatcgtggggaataaggacagtgtcacccgggggatccggtggtggtgcaaatcaaagaactgctcctcagtggatgttgcctttacttctaggcctgtacggaagtgttacttctgctctaaaagctgcggaattgtacccgcggccgatccaccggtcgccaccatctagcatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactagctcgacgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 12 - DNA, 2285 bp, Artificial Sequence
  Synthetic
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcggtacaattcacgcgtactagtaggttaatttttaaaaagcagtcaaaagtccaagtggcccttggcagcatttactctctctgtttgctctggttaataatctcaggagcacaaacattccgctagcagatccaggttaatttttaaaaagcagtcaaaagtccaagtggcccttggcagcatttactctctctgtttgctctggttaataatctcaggagcacaaacattccggtaccccatgggggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatatttgtggcggccctagagtcgatcgaggaactgaaaaaccagaaagttaactggtaagtttagtctttttgtcttttatttcaggtcccagatcagggctctgcgtttgctccaggtagtccgctgctcccttgggcctgggcccactgacagccctggtgcctctggccggctgcacacctcctggcgggcagctgtgtttaaggtcactcggatggactgttctggcaatacctggtccatccctgcgaccttaaacacggaggcctgccctgactgcccacggtgccgtggccaaagaggatctaagggcaccgctgagggcctacctaaccatcgtggggaataaggacagtgtcacccgggggatccggtggtggtgcaaatcaaagaactgctcctcagtggatgttgcctttacttctaggcctgtacggaagtgttacttctgctctaaaagctgcggaattgtacccgcggccgatccaccggtcgccaccatctagcatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactagctcgacgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag